(19) United States
(12) Patent Application Publication
(10) Pub. No.: US 2005/0003474 A1
(43) Pub. Date: Jan. 6, 2005

(54) Carotenoid biosynthesis

(76) Inventors: Mervyn L. Desouza, Plymouth, MN (US); Sherry R. Kollmann, Maple Grove, MN (US)

Correspondence Address: CARGILL, INCORPORATED, LAW/24, 15407 MCGINTY ROAD WEST, WAYZATA, MN, 55391, US

(21) Appl. No.: 10/848,307

(22) Filed: May 18, 2004

Related U.S. Application Data
(63) Continuation-in-part of application No. 10/466,656, filed on Jul. 18, 2003.
(60) Provisional application No. 60/264,329, filed on Jan. 26, 2001.
(60) Provisional application No. 60/288,984, filed on May 4, 2001.

(51) Int. Cl. C12P 23/00; C12N 9/10; C07H 21/04; C12N 1/21

(52) U.S. Cl. 435/67; 435/69.1; 435/252.3; 435/193; 536/23.2

(57) ABSTRACT

Membranous bacteria that produce astaxanthin and other carotenoids are described, as well as isolated nucleic acids and expression vectors that can be used for producing carotenoids in microorganisms.

### CROSS REFERENCE TO RELATED APPLICATIONS

This application is a continuation-in-part of co-pending U.S. Ser. No. 10/466,656, filed Jul. 18, 2003, which claims the benefit of application Ser. No. PCT/US02/02124, filed Jan. 25, 2002 which claims the benefit of U.S. Provisional Application Ser. Nos. 60/288,984, filed May 4, 2001 and 60/264,329 filed Jan. 25, 2001.

### TECHNICAL FIELD

The invention relates to methods and materials for producing carotenoids, and in particular, to nucleic acid molecules, polypeptides, host cells, and methods that can be used for producing carotenoids.

### BACKGROUND

Astaxanthin (3,3′-dihydroxy-β,β-carotene-4,4′-dione) is the primary carotenoid that imparts the pink pigment to the eggs, flesh, and skin of salmon, trout, and shrimp. Most animals cannot synthesize carotenoids. Rather, the pigments are acquired through the food chain from marine algae and phytoplankton, the primary producers of astaxanthin. ATX exists in three configurational isomers [(3S, 3′S), (3R, 3′R) and (3S, 3′R; 3R, 3′S)], however, ATX is found in the marine environment only in the (3S, 3′S) form. Consequently, this form is considered the natural and most desirable form of ATX.

Although astaxanthin has been commercially extracted from some yeast and crustacea species and has been chemically synthesized as a 1:2:1 mixture of the (3S,3′S)-, (3S,3′R)- and (3R,3′R)-isomers, astaxanthin is limited in availability and is expensive to purchase. See, Torrisen et al. (1989) Crit. Rev. Aquatic Sci. 1:209; and Mayer (1994) Pure Appl. Chem., 66:931-938. Thus, there is a need for a less expensive source of the naturally-occurring (3S,3′S) astaxanthin.

### SUMMARY

The invention is based on methods and materials for producing carotenoids such as lycopene, zeaxanthin, zeaxanthin diglucoside, canthaxanthin, β-carotene, lutein, and astaxanthin. Such carotenoids can be used as nutritional supplements in humans and can be formulated for use in aquaculture or as an animal feed. The invention provides nucleic acid molecules that can be used to engineer host cells having the ability to produce particular carotenoids and polypeptides that can be used in cell-free systems to make particular carotenoids. The engineered cells described herein can be used to produce large quantities of carotenoids.

In one aspect, the invention features an isolated nucleic acid having at least 76% sequence identity to the nucleotide sequence of SEQ ID NO: 1 (e.g., at least 80%, 85%, 90%, or 95% sequence identity to the nucleotide sequence of SEQ ID NO: 1) or to a fragment of SEQ ID NO:1 at least 33 contiguous nucleotides in length. An isolated nucleic acid can encode a zeaxanthin glucosyl transferase polypeptide at least 75% identical to the amino acid sequence of SEQ ID NO:2. Expression vectors containing such nucleic acids operably linked to an expression control element also are featured.

In another aspect, the invention features an isolated nucleic acid having at least 78% sequence identity to the nucleotide sequence of SEQ ID NO:3 (e.g., at least 80%, 85%, 90%, or 95% sequence identity to the nucleotide sequence of SEQ ID NO:3) or to a fragment of SEQ ID NO:3 at least 32 contiguous nucleotides in length. An isolated nucleic acid can encode a lycopene β-cyclase polypeptide at least 83% identical to the amino acid sequence of SEQ ID NO:4. β-carotene can be made by contacting lycopene with a polypeptide encoded by such isolated nucleic acids. The invention also features an expression vector that includes such nucleic acids operably linked to an expression control element.

In yet another aspect, the invention features an isolated nucleic acid having at least 81% sequence identity to the nucleotide sequence of SEQ ID NO:5 (e.g., at least 85%, 90%, or 95% sequence identity to the nucleotide sequence of SEQ ID NO:5) or to a fragment of SEQ ID NO:5 at least 60 contiguous nucleotides in length. An isolated nucleic acid also can encode a geranylgeranyl pyrophosphate synthase polypeptide at least 85% identical to the amino acid sequence of SEQ ID NO:6. Geranylgeranyl pyrophosphate can be made by contacting farnesyl pyrophosphate and isopentenyl pyrophosphate with a polypeptide encoded by such nucleic acids. Expression vectors that include such nucleic acids operably linked to an expression control element also are featured.

Isolated nucleic acids having at least 82% sequence identity to the nucleotide sequence of SEQ ID NO:7 (e.g., at least 85%, 90%, or 95% sequence identity to the nucleotide sequence of SEQ ID NO:7) or to a fragment of SEQ ID NO:7 at least 30 contiguous nucleotides in length also are featured. An isolated nucleic acid also can encode a phytoene desaturase polypeptide at least 90% identical to the amino acid sequence of SEQ ID NO:8. Lycopene can be made by contacting phytoene with a polypeptide encoded by such nucleic acids. An expression vector that includes such nucleic acids operably linked to an expression control element also is featured.

The invention also features an isolated nucleic acid having at least 82% sequence identity to the nucleotide sequence of SEQ ID NO:9 (e.g., at least 85%, 90%, or 95% sequence identity to the nucleotide sequence of SEQ ID NO:9) or to a fragment of SEQ ID NO:9 at least 23 contiguous nucleotides in length. An isolated nucleic acid also can encode a phytoene synthase polypeptide at least 89% identical to the amino acid sequence of SEQ ID NO:10. Phytoene can be made by contacting geranylgeranyl pyrophosphate with a polypeptide encoded by such nucleic acids. An expression vector that includes such nucleic acids operably linked to an expressioni control element also is featured.

In yet another aspect, the invention features an isolated nucleic acid having at least 85% sequence identity to the nucleotide sequence of SEQ ID NO:11 (e.g., at least 90% or 95% identity to the nucleotide sequence of SEQ ID NO:11) or to a fragment of SEQ ID NO:11 at least 36 contiguous nucleotides in length. An isolated nucleic acid can encode a β-carotene hydroxylase polypeptide at least 90% identical to the amino acid sequence of SEQ ID NO:12. Zeaxanthin can be made by contacting β-carotene with a polypeptide encoded by such nucleic acids. Astaxanthin can be made by contacting canthaxanthin with a polypeptide encoded by such nucleic acids. The invention also features an expression vector that includes such nucleic acids operably linked to an expression control element.

The invention also features membranous bacteria (e.g., a Rhodobacter species) that include at least one exogenous nucleic acid encoding phytoene desaturase, lycopene β-cyclase, β-carotene hydroxylase, and β-carotene C4 oxygenase, wherein expression of the at least one exogenous nucleic acid produces detectable amounts of astaxanthin in the membranous bacteria. The amino acid sequence of the phytoene desaturase can be at least 90% identical to the amino acid sequence of SEQ ID NO:8. The amino acid sequence of the lycopene β-cyclase can be at least 83% identical to the amino acid sequence of SEQ ID NO:4. The amino acid sequence of the β-carotene hydroxylase can be at least 90% identical to the amino acid sequence of SEQ ID NO:12. The amino acid sequence of the β-carotene C4 oxygenase can be at least 80% identical to the amino acid sequence of SEQ ID NO:39. The membranous bacteria further can include an exogenous nucleic acid encoding geranylgeranyl pyrophosphate synthase (e.g., a multifunctional geranylgeranyl pyrophosphate synthase) or can lack endogenous bacteriochlorophyll biosynthesis. The multifunctional geranylgeranyl pyrophosphate synthase can have an amino acid sequence at least 90% identical to the amino acid sequence of SEQ ID NO:45. The membranous bacteria further can include an exogenous nucleic acid encoding phytoene synthase. The phytoene synthase can have an amino acid sequence at least 89% identical to the amino acid sequence of SEQ ID NO:10.

In another aspect, the invention features membranous bacteria that include an exogenous nucleic acid encoding a phytoene desaturase having an amino acid sequence at least 90% identical to the amino acid sequence of SEQ ID NO:8, and wherein the membranous bacteria produces detectable amounts of lycopene. The membranous bacteria further can include a lycopene β-cyclase, wherein the membranous bacteria produce detectable amounts of β-carotene. The membranous bacteria also can include a β-carotene hydroxylase, wherein the membranous bacteria produce detectable amounts of zeaxanthin.

In still yet another aspect, the invention feature membranous bacteria that include at least one exogenous nucleic acid encoding phytoene desaturase, lycopene β-cyclase, and β-carotene C4 oxygenase, wherein expression of the at least one exogenous nucleic acid produces detectable amounts of canthaxanthin in the membranous bacteria. The membranous bacteria also can include a β-carotene hydroxylase, wherein the membranous bacteria produce detectable amounts of astaxanthin.

The invention also features a composition that includes an engineered Rhodobacter cell, wherein the cell produces a detectable amount of astaxanthin or canthaxanthin. The engineered Rhodobacter cell can include at least one exogenous nucleic acid encoding phytoene desaturase, lycopene β-cyclase, β-carotene hydroxylase, and β-carotene C4 oxygenase. The composition can be formulated for aquaculture and can pigment the flesh of fish or the carapace of crustaceans after ingestion. The composition can be formulated for human consumption or as an animal feed (e.g., formulated for consumption by chickens, turkeys, cattle, swine, or sheep).

The invention also features a method of making a nutraceutical. The method includes extracting carotenoids from an engineered Rhodobacter cell, the engineered Rhodobacter cell including at least one exogenous nucleic acid encoding phytoene desaturase, lycopene β-cyclase, β-carotene hydroxylase, and β-carotene C4 oxygenase, and wherein the Rhodobacter cell produces detectable amounts of astaxanthin.

In yet another aspect, the invention features membranous bacteria, wherein the membranous bacteria include an exogenous nucleic acid encoding a lycopene β-cyclase having an amino acid sequence at least 83% identical to the amino acid sequence of SEQ ID NO:4. The membranous bacteria further can include a phytoene desaturase, (e.g., an exogenous phytoene desaturase), wherein the membranous bacteria produce detectable amounts of β-carotene. The membranous bacteria also can include a β-carotene hydroxylase (e.g., an exogenous β-carotene hydroxylase), wherein the bacteria produce detectable amounts of zeaxanthin.

Membranous bacteria that include a β-carotene hydroxylase having an amino acid sequence at least 90% identical to the amino acid sequence of SEQ ID NO:12 also is featured. The membranous bacteria further can include a lycopene β-cyclase (e.g., an exogenous lycopene β-cyclase), wherein the membranous bacteria produce detectable amounts of zeaxanthin. The membranous bacteria also can include a phytoene desaturase (e.g., an exogenous phytoene desaturase), wherein the membranous bacteria produce detectable amounts of β-carotene.

The invention also features membranous bacteria (e.g., a Rhodobacter species) lacking an endogenous nucleic acid encoding a famesyl pyrophosphate synthase, wherein the bacteria produces detectable amounts of carotenoids. The membranous bacteria also can include an exogenous nucleic acid encoding a multifunctional geranylgeranyl pyrophosphate synthase.

In another aspect, the invention features an isolated nucleic acid having at least 70% sequence identity (e.g., at least 80% or 90%) to the nucleotide sequences of SEQ ID NO:38, or to a fragment of the nucleic acid of SEQ ID NO:38 at least 15 contiguous nucleotides in length. The nucleic acid can encode a β-carotene C4 oxygenase. Canthaxanthin can be made by contacting β-carotene with a polypeptide encoded by such nucleic acids or a polypeptide having an amino acid sequence at least 80% identical to the amino acid sequence of SEQ ID NO:39. Astaxanthin can be made by contacting zeaxanthin with a polypeptide encoded by such isolated nucleic acids or a polypeptide having an amino acid sequence at least 80% identical to the amino acid sequence of SEQ ID NO:39.

In another aspect, the invention features membranous bacteria that include an exogenous nucleic acid encoding a β-carotene C4 oxygenase, where the β-carotene oxygenase has an amino acid sequence at least 80% identical to the amino acid sequence of SEQ ID NO:39.

In yet another aspect, the invention features a host cell comprising an exogenous nucleic acid, wherein the exogenous nucleic acid includes a nucleic acid sequence encoding one or more polypeptides that catalyze the formation of (3S, 3′S) astaxanthin, wherein the host cell produces CoQ-10 and (3S, 3′S) astaxanthin. A method of making CoQ-10 and (3S, 3′S) astaxanthin at substantially the same time also is featured. The method includes transforming a host cell with a nucleic acid, wherein the nucleic acid includes a nucleic acid sequence that encodes one or more polypeptides, wherein the polypeptides catalyze the formation of (3S, 3′S) astaxanthin; and culturing the host cell under conditions that allow for the production of (3S, 3′S) astaxanthin and CoQ-10. The method further can include transforming the host cell with at least one exogenous nucleic acid,-the exogenous nucleic acid encoding one or more polypeptides, wherein the polypeptides catalyze the formation of CoQ-10.

The invention also features isolated nucleic acid having a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:38, and SEQ ID NO:44.

An isolated nucleic acid having at least 90% sequence identity to the nucleotide sequences of SEQ ID NO:44, or to a fragment of the nucleic acid of SEQ ID NO:44 at least 60 contiguous nucleotides in length is featured. Geranylgeranyl pyrophosphate can be made by contacting isopentenyl pyrophosphate and dimethylallyl pyrophosphate with a polypeptide encoded by such a nucleic acid.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic diagram of the biosynthetic pathway for the production of zeaxanthin and conversion to zeaxanthin di-glucoside.

FIG. 2 is a schematic diagram of the P. stewartii carotenoid gene operon (6586 bp).

FIG. 3 is a chromatogram of astaxanthin production in P. stewartii::crtW(B. aurantiaca).

FIG. 4 is a schematic diagram of the biosynthetic pathway leading to neurosporene. The diagram indicates how the disablement of the hydroxyneurosporene gene (crtC) relates to the pathways that eventually produce, lycopene, beta-carotein, zeaxanthin, decapreoxanthin, lutein, and astaxanthin. More detailed descriptions of pathways that produce these carotenoids are provided in PCT/US01/43906 and PCT/US01/07178, which are herein incorporated by reference.

### DETAILED DESCRIPTION

Nucleic Acid Molecules

The invention features isolated nucleic acids that encode enzymes involved in carotenoid biosynthesis. The nucleic acids of SEQ ID NO:1, 3, 5, 7, 9, and 11 encode zeaxanthin glucosyl transferase (crtX), lycopene β-cyclase (crtY), geranylgeranyl-pyrophosphate synthase (crtE), phytoene desaturase (crtI), phytoene synthase (crtB) and β-carotene hydroxylase (crtZ), respectively. A nucleic acid of the invention can have at least 76% sequence identity, e.g., 78%, 80%, 85%, 90%, 95%, or 99% sequence identity, to the nucleic acid of SEQ ID NO:1, or to fragments of the nucleic acid of SEQ ID NO:1 that are at least about 33 nucleotides in length; at least 78% sequence identity, e.g., 80%, 85%, 90%, 95%, or 99% sequence identity, to the nucleotide sequence of SEQ ID NO:3, or to fragments of the nucleic acid of SEQ ID NO:3 that are at least about 32 nucleotides in length; at least 81% sequence identity, e.g., 82%, 85%, 90%, 95%, or 99% sequence identity, to the nucleotide sequence of SEQ ID NO:5 , or to fragments of the nucleic acid of SEQ ID NO:5 that are at least about 60 nucleotides in length; at least 82% sequence identity, e.g., 83%, 85%, 90%, 95%, or 99% sequence identity, to the nucleotide sequences of SEQ ID NO:7 or SEQ ID NO:9, or to fragments of the nucleic acids of SEQ ID NO:7 or SEQ ID NO:9 that are at least about 30 or 23 nucleotides in length, respectively; at least 85% sequence identity, e.g., 86%, 90%, 92%, 95%, or 99% sequence identity, to the nucleotide sequence of SEQ ID NO:11 , or to fragments of the nucleic acid of SEQ ID NO:11 that are at least about 36 nucleotides in length. A nucleic acid of the invention can have at least 60% sequence identity, e.g., at least 65%, 70%, 75%, 80%, 85%, 90%, 95%, or 99% sequence identity to the nucleotide sequence of SEQ ID NO:38 or to fragments of the nucleic acid of SEQ ID NO:38 that are at least about 15 nucleotides in length. Such a nucleic acid can encode a β-carotene C4 oxygenase (crtW). A nucleic acid of the invention also can have at least 90% identity to the nucleotide sequence set forth in SEQ ID NO:44 or to fragments of the nucleic acid of SEQ ID NO:44 that are at least about 60 nucleotides in length. Such a nucleic acid can encode a multifunctional geranylgeranyl pyrophosphate synthase.

Generally, percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity can be determined for any nucleic acid or amino acid sequence as follows. First, a nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from the University of Wisconsin library as well as at www.fr.com or www.ncbi.nlm.nih.gov. Instructions explaining how to use the Bl2seq program can be found in the readme file accompanying BLASTZ.

Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:seq1.txt); -j is set to a file containing the second nucleic acid sequence to be compared (e.g., C:seq2.txt); -p is set to blastn; -o is set to any desired file name (e.g., C:output.txt); -q is set to −1; -r is set to 2; and all other options are left at their default setting. For example, the following command can be used to generate an output file containing a comparison between two sequences: C:Bl2seq -i c:seq1.txt -j c:seq2.txt -p blastn -o c:output.txt -q-1-r2. To compare two amino acid sequences, the options of Bl2seq are set as follows: -i is set to a file containing the first amino acid sequence to be compared (e.g., C:seq1.txt); -j is set to a file containing the second amino acid sequence to be compared (e.g., C:seq2.txt); -p is set to blastp; -o is set to any desired file name (e.g., C:output.txt); and all other options are left at their default setting. For example, the following command can be used to generate an output file containing a comparison between two amino acid sequences: C:Bl2seq -i c:seq1.txt -j c:seq2.txt -p blastp -o c:output.txt. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides or amino acid residues from the target sequence presented in alignment with sequence from the identified sequence starting with any matched position and ending with any other matched position. A matched position is any position where an identical nucleotide or amino acid residue is presented in both the target and identified sequence. Gaps presented in the target sequence are not counted since gaps are not nucleotides or amino acid residues. Likewise, gaps presented in the identified sequence are not counted since target sequence nucleotides or amino acid residues are counted, not nucleotides or amino acid residues from the identified sequence.

The percent identity over a particular length is determined by counting the number of matched positions over that length and dividing that number by the length followed by multiplying the resulting value by 100. For example, if (1) a 1000 nucleofide target sequence is compared to the sequence set forth in SEQ ID NO:1, (2) the Bl2seq program presents 200 nucleotides from the target sequence aligned with a region of the sequence set forth in SEQ ID NO:1 where the first and last nucleotides of that 200 nucleotide region are matches, and (3) the number of matches over those 200 aligned nucleotides is 180, then the 1000 nucleotide target sequence contains a length of 200 and a percent identity over that length of 90 (i.e. 180÷200*100=90).

It will be appreciated that a single nucleic acid or amino acid target sequence that aligns with an identified sequence can have many different lengths with each length having its own percent identity. For example, a target sequence containing a 20 nucleotide region that aligns with an identified sequence as follows has many different lengths including those listed in Table 1.

| | | |
|---|---|---|
| | 1 20 | |
| Target Sequence: | AGGTCGTGTACTGTCAGTCA | (SEQ ID NO:46) |
| | \| \|\| \|\|\| \|\|\|\| \|\|\|\| \| | |
| Identified Sequence: | ACGTGGTGAACTGCCAGTGA | (SEQ ID NO:47) |

TABLE 1

| Starting | Ending | | Matched | Percent |
|---|---|---|---|---|
| Position | Position | Length | Positions | Identity |
| 1 | 20 | 20 | 15 | 75.0 |
| 1 | 18 | 18 | 14 | 77.8 |
| 1 | 15 | 15 | 11 | 73.3 |
| 6 | 20 | 15 | 12 | 80.0 |
| 6 | 17 | 12 | 10 | 83.3 |
| 6 | 15 | 10 | 8 | 80.0 |
| 8 | 20 | 13 | 10 | 76.9 |
| 8 | 16 | 9 | 7 | 77.8 |

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

Isolated nucleic acid molecules of the invention are at least about 20 nucleotides in length. For example, the nucleic acid molecule can be about 20-30, 22-32, 33-50, 34 to 45, 40-50, 60-80, 62 to 92, 50-100, or greater than 150 nucleotides in length, e.g., 200-300, 300-500, or 500-1000 nucleotides in length. Such fragments, whether protein-encoding or not, can be used as probes, primers, and diagnostic reagents. In some embodiments, the isolated nucleic acid molecules encode a full-length zeaxanthin glucosyl transferase, lycopene β-cyclase, geranylgeranyl pyrophosphate synthase, phytoene desaturase, β-carotene hydroxylase, β-carotene C4 oxygenase, or multifunctional geranylgeranyl pyrophosphate synthase polypeptide. Nucleic acid molecules can be DNA or RNA, linear or circular, and in sense or antisense orientation.

Isolated nucleic acid molecules of the invention can be produced by standard techniques. As used herein, “isolated” refers to a sequence corresponding to part or all of a gene encoding a zeaxanthin glucosyl transferase, lycopene β-cyclase, geranylgeranyl-pyrophosphate synthase, phytoene desaturase, phytoene synthase, β-carotene hydroxylase, β-carotene C4 oxygenase, or multifunctional geranylgeranyl pyrophosphate synthase polypeptide, or an operon encoding two or more such polypeptides, but free of sequences that normally flank one or both sides of the wild-type gene or the operon in a naturally-occurring genome, e.g., a bacterial genome. The term “isolated” as used herein with respect to nucleic acids also includes any non-naturally-occurring nucleic acid sequence since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a cDNA or genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acids within the scope of the invention can be obtained using any method including, without limitation, common molecular cloning and chemical nucleic acid synthesis techniques. For example, polymerase chain reaction (PCR) techniques can be used to obtain an isolated nucleic acid containing a nucleic acid sequence sharing identity with the sequences set forth in SEQ ID NOs: 1, 3, 5, 7, 9, 11, 38, or 44. PCR refers to a procedure or technique in which target nucleic acids are amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, Ed. by Dieffenbach, C. and Dveksler, G., Cold Spring Harbor Laboratory Press, 1995. When using RNA as a source of template, reverse transcriptase can be used to synthesize complimentary DNA (cDNA) strands.

Isolated nucleic acids of the invention also can be chemically synthesized, either as a single nucleic acid molecule or as a series of oligonucleotides. For example, one or more pairs of long oligonucleotides (e.g., >100 nucleotides) can be synthesized that contain the desired sequence, with each pair containing a short segment of complementary (e.g., about 15 nucleotides) DNA such that a duplex is formed when the oligonucleotide pair is annealed. DNA polymerase is used to extend the oligonucleotides, resulting in a double-stranded nucleic acid molecule per oligonucleotide pair, which then can be ligated into a vector.

Isolated nucleic acids of the invention also can be obtained by mutagenesis. For example, an isolated nucleic acid that shares identity with a sequence set forth in SEQ ID NO:1, 3, 5, 7, 9, 11, 38, or 44 can be mutated using common molecular cloning techniques (e.g., site-directed mutagenesis). Possible mutations include, without limitation, deletions, insertions, and substitutions, as well as combinations of deletions, insertions, and substitutions. Alignments of nucleic acids of the invention with other known sequences encoding carotenoid enzymes can be used to identify positions to modify. For example, alignment of the nucleotide sequence of SEQ ID NO:5 with other nucleic acids encoding geranyl geranyl pyrophosphate synthases (e.g., from Erwinia uredovora) provides guidance as to which nucleotides can be substituted, which nucleotides can be deleted, and at which positions nucleotides can be inserted.

In addition, nucleic acid and amino acid databases (e.g., GenBank®) can be used to obtain an isolated nucleic acid within the scope of the invention. For example, any nucleic acid sequence having homology to a sequence set forth in SEQ ID NO:1, 3, 5, 7, 9, 11, 38, or 44, or any amino acid sequence having homology to a sequence set forth in SEQ ID NO:2, 4, 6, 8, 10, 12, 39, or 45 can be used as a query to search GenBank®.

Furthermore, nucleic acid hybridization techniques can be used to obtain an isolated nucleic acid within the scope of the invention. Briefly, any nucleic acid having some homology to a sequence set forth in SEQ ID NO:1, 3, 5, 7, 9, 11, 38, or 44 can be used as a probe to identify a similar nucleic acid by hybridization under conditions of moderate to high stringency. Moderately stringent hybridization conditions include hybridization at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), and wash steps at about 50° C. with a wash solution containing 2×SSC and 0.1% SDS. For high stringency, the same hybridization conditions can be used, but washes are performed at about 65° C. with a wash solution containing 0.2×SSC and 0.1% SDS.

Once a nucleic acid is identified, the nucleic acid then can be purified, sequenced, and analyzed to determine whether it is within the scope of the invention as described herein. Hybridization can be done by Southern or Northern analysis to identify a DNA or RNA sequence, respectively, that hybridizes to a probe. The probe can be labeled with biotin, digoxygenin, an enzyme, or a radioisotope such as ³P or ³⁵S. The DNA or RNA to be analyzed can be electrophoretically separated on an agarose or polyacrylamide gel, transferred to nitrocellulose, nylon, or other suitable membrane, and hybridized with the probe using standard techniques well known in the art. See, for example, sections 7.39-7.52 of Sambrook et al., (1989) Molecular Cloning, second edition, Cold Spring harbor Laboratory, Plainview, N.Y.

Polypeptides

The present invention also features isolated zeaxanthin glucosyl transferase (SEQ ID NO:2), lycopene β-cyclase (SEQ ID NO:4), geranylgeranyl pyrophosphate synthase (SEQ ID NO:6), phytoene desaturase (SEQ ID NO:8), phytoene synthase (SEQ ID NO:10), and β-carotene hydroxylase (SEQ ID NO:12) polypeptides. In addition, the invention features isolated β-carotene C4 oxygenase polypeptides (SEQ ID NO:39) and multifunctional geranylgeranyl pyrophosphate synthase polypeptides (SEQ ID NO:45). A polypeptide of the invention can have at least 75% sequence identity, e.g., 80%, 85%, 90%, 95%, or 99% sequence identity, to the amino acid sequence of SEQ ID NO:2 or to fragments thereof; at least 83% sequence identity, e.g., 85%, 90%, 95%, or 99% sequence identity, to the amino acid sequence of SEQ ID NO:4 or to fragments thereof; at least 85% sequence identity, e.g., 90%, 95%, or 99% sequence identity, to the amino acid sequence of SEQ ID NO:6 or to fragments thereof; at least 90% sequence identity, e.g., 90%, 92%, 95%, or 99% sequence identity, to the amino acid sequence of SEQ ID NO:8 or to fragments thereof; at least 89% sequence identity, e.g., 90%, 95%, or 99% sequence identity, to the amino acid sequence of SEQ ID NO:10 or to fragments thereof; at least 90% sequence identity, e.g., 95%, or 99% sequence identity, to the amino acid sequence of SEQ ID NO:12 or to fragments thereof; at least 60% sequence identity, e.g., 65%, 70%, 75%, 80%, 85%, 90%, 95%, or 99% sequence identity, to the amino acid sequence of SEQ ID NO:39 or to fragments thereof; or at least 90% sequence identity, e.g., 95% or 99% sequence identity, to the amino acid sequence set forth in SEQ ID NO:45 or to fragments thereof. Percent sequence identity can be determined as described above for nucleic acid molecules.

An “isolated polypeptide” has been separated from cellular components that naturally accompany it. Typically, the polypeptide is isolated when it is at least 60% (e.g., 70%, 80%, 90%, 95%, or 99%), by weight, free from proteins and naturally-occurring organic molecules that are naturally associated with it. In general, an isolated polypeptide will yield a single major band on a non-reducing polyacrylamide gel.

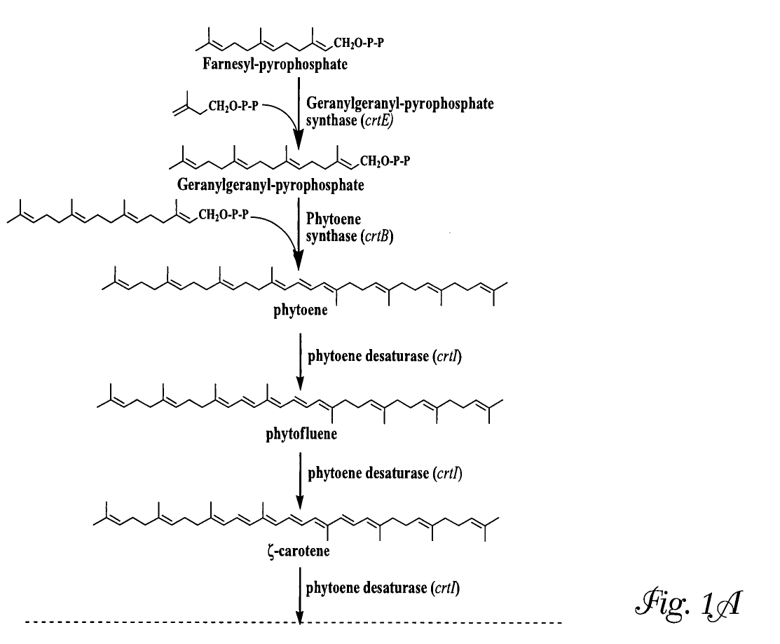

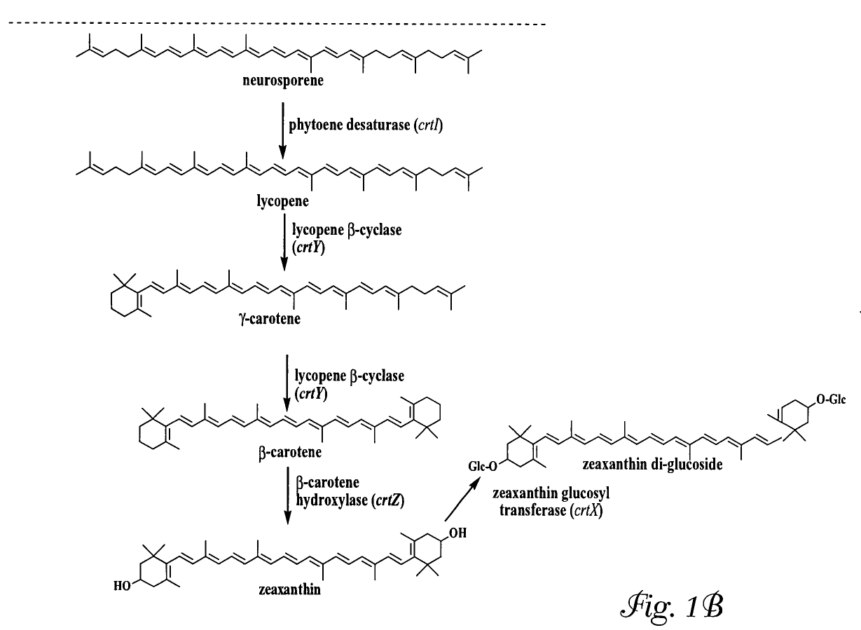

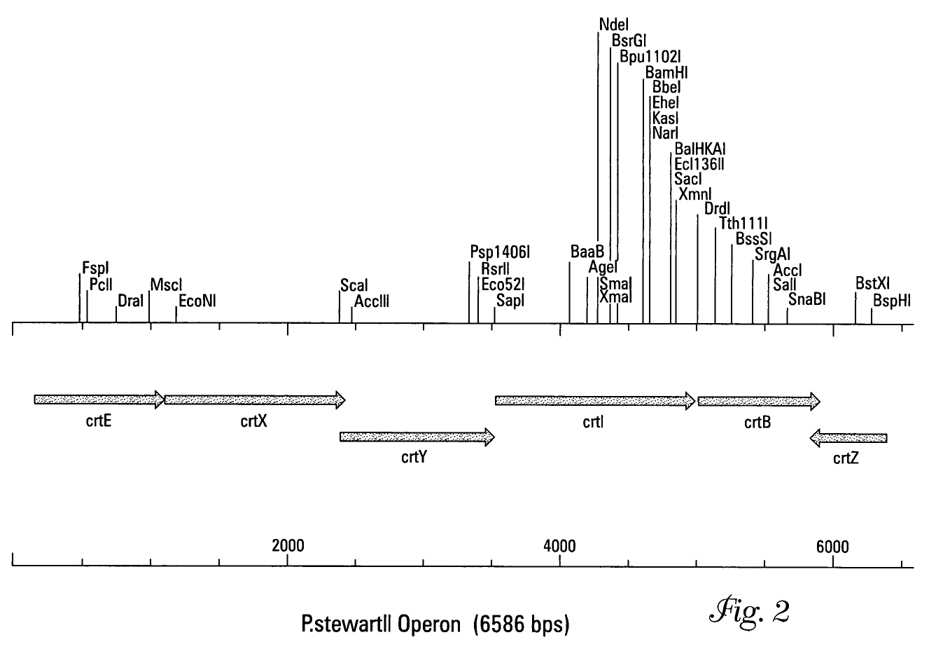

The term “polypeptide” includes any chain of amino acids, regardless of length or post-translational modification. Polypeptides that have identity to the amino acid sequences of SEQ ID NO:2, 4, 6, 8, 10, 12, 39, or 45 can retain the function of the enzyme (see FIG. 1 for a schematic of the carotenoid biosynthesis pathway). For example, geranylgeranyl pyrophosphate synthase can produce geranylgeranyl pyrophosphate (GGPP) by condensing together isopentenyl pyrophosphate (IPP) with farnesyl pyrophosphate (FPP). Phytoene synthase can produce phytoene by condensing together two molecules of GGPP. Phytoene desaturase can perform four successive desaturations on phytoene to form lycopene. Lycopene β-cyclase can perform two successive cyclization reactions on lycopene to form β-carotene. β-carotene hydroxylase can perform two successive hydroxylation reactions on β-carotene to form zeaxanthin. Alternatively, β-carotene hydroxylase can perform two successive hydroxylation reactions on canthaxanthin to form astaxanthin. Zeaxanthin glucosyl transferase can add one or two glucose or other sugar moieties to zeaxanthin to form zeaxanthin monoglycoside or diglycoside, respectively. β-carotene C4 oxygenase can convert the methylene groups at the C4 and C4′ positions of the β-carotene or zeaxanthin to form canthaxanthin or astaxanthin, respectively. Multifunctional geranylgeranyl pyrophosphate synthase can directly convert 3 IPP molecules and 1 dimethylallyl pyrophosphate (DMAPP) molecule to 1 GGPP molecule.

In general, conservative amino acid substitutions, i.e., substitutions of similar amino acids, are tolerated without affecting protein function. Similar amino acids are those that are similar in size and/or charge properties. Families of amino acids with similar side chains are known. These families include amino acids with basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), β-branched side chains (e.g., threonine, valine, or isoleucine), and aromatic side chains (e.g., tyrosine, phenylalanine, tryptophan, or histidine).

Mutagenesis also can be used to alter a nucleic acid such that activity of the polypeptide encoded by the nucleic acid is altered (e.g., to increase production of a particular carotenoid). For example, error-prone PCR (e.g., (GeneMorph PCR Mutagenesis Kit; Stratagene Inc. La Jolla, Calif.; Catalog #600550; Revision #090001) can be used to mutagenize the B. aurantiaca crtW gene (SEQ ID NO:38) to increase the relative amount of di-keto carotenoid (e.g. astaxanthin (3,3′-dihydroxy-β,β-carotene-4,4′-dione) or canthaxanthin (β,β-carotene4,4′-dione)) relative to mono-keto carotenoid (e.g. echinone (β,β-carotene4-one) or adonixanthin (3,3′-dihydroxy-β,β-carotene-4-one)) that is produced. In general, the nucleic acid to be mutagenized can be cloned into a vector such as pCR-Blunt II-TOPO (Clontech; Palo Alto, Calif.) and used as a template for error-prone PCR. For purposes of directed evolution, mutation frequencies of 2-7 nucleotides/Kbp template (14 amino acids mutations/333 Amino acids) generally are desired. Mutation frequency can be lowered or raised by increasing or decreasing the template concentration, respectively. PCR can be performed according to manufacturer's recommendations. Mutagenized nucleic acid is ligated into an expression vector, which is used to transform a host, and activity of the expressed protein is assessed. For example, in the case of the crt W gene, electrocompetent P. stewartii (ATCC 8200) cells can be prepared and transformed as described herein, and resulting individual colonies can be screened by visual inspection for a phenotypic change from bright yellow pigmentation (production of zeaxanthin), yellow orange (production of mono-keto carotenoid) or reddish-orange (production of di-keto carotenoid). Production of increased amounts of astaxanthin can be confirmed by HPLC/MS.

Isolated polypeptides of the invention can be obtained, for example, by extraction from a natural source (e.g., a plant or bacteria cell), chemical synthesis, or by recombinant production in a host. For example, a polypeptide of the invention can be produced by ligating a nucleic acid molecule encoding the polypeptide into a nucleic acid construct such as an expression vector, and transforming a bacterial or eukaryotic host cell with the expression vector. In general, nucleic acid constructs include expression control elements operably linked to a nucleic acid sequence encoding a polypeptide of the invention (e.g., zeaxanthin glucosyl transferase, lycopene β-cyclase, geranylgeranyl pyrophosphate synthase, phytoene desaturase, phytoene synthase, β-carotene hydroxylase, β-carotene C4 oxygenase, or multifunctional geranylgeranyl pyrophosphate synthase polypeptides). Expression control elements do not typically encode a gene product, but instead affect the expression of the nucleic acid sequence. As used herein, “operably linked” refers to connection of the expression control elements to the nucleic acid sequence in such a way as to permit expression of the nucleic acid sequence. Expression control elements can include, for example, promoter sequences, enhancer sequences, response elements, polyadenylation sites, or inducible elements. Non-limiting examples of promoters include the puf promoter from Rhodobacter sphaeroides (GenBank Accession No. E13945), the nifHDK promoter from R. sphaeroides (GenBank Accession No. AF031817), and the fliK promoter from R. sphaeroides (GenBank Accession No. U86454).

In bacterial systems, a strain of E. coli such as DH10B or BL-21 can be used. Suitable E. coli vectors include, but are not limited to, pUC18, pUC19, the pGEX series of vectors that produce fusion proteins with glutathione S-transferase (GST), and pBluescript series of vectors. Transformed E. coli are typically grown exponentially then stimulated with isopropylthiogalactopyranoside (IPTG) prior to harvesting. In general, fusion proteins produced from the pGEX series of vectors are soluble and can be purified easily from lysed cells by adsorption to glutathione-agarose beads followed by elution in the presence of free glutathione. The pGEX vectors are designed to include thrombin or factor Xa protease cleavage sites such that the cloned target gene product can be released from the GST moiety.

In eukaryotic host cells, a number of viral-based expression systems can be utilized to express polypeptides of the invention. A nucleic acid encoding a polypeptide of the invention can be cloned into, for example, a baculoviral vector such as pBlueBac (Invitrogen, San Diego, Calif.) and then used to co-transfect insect cells such as Spodoptera frugiperda (Sf9) cells with wild-type DNA from Autographa californica multiply enveloped nuclear polyhedrosis virus (AcMNPV). Recombinant viruses producing polypeptides of the invention can be identified by standard methodology. Alternatively, a nucleic acid encoding a polypeptide of the invention can be introduced into a SV40, retroviral, or vaccinia based viral vector and used to infect suitable host cells.

A polypeptide within the scope of the invention can be “engineered” to contain an amino acid sequence that allows the polypeptide to be captured onto an affinity matrix. For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini. Other fusions that could be useful include enzymes that aid in the detection of the polypeptide, such as alkaline phosphatase.

Agrobacterium-mediated transformation, electroporation and particle gun transformation can be used to transform plant cells. Illustrative examples of transformation techniques are described in U.S. Pat. No. 5,204,253 (particle gun) and U.S. Pat. No. 5,188,958 (Agrobacterium). Transformation methods utilizing the Ti and Ri plasmids of Agrobacterium spp. typically use binary type vectors. Walkerpeach, C. et al., in Plant Molecular Biology Manual, S. Gelvin and R. Schilperoort, eds., Kluwer Dordrecht, C 1:1-1 9 (1994). If cell or tissue cultures are used as the recipient tissue for transformation, plants can be regenerated from transformed cultures by techniques known to those skilled in the art.

Engineered Cells

Any cell containing an isolated nucleic acid within the scope of the invention is itself within the scope of the invention. This includes, without limitation, prokaryotic cells such as R. sphaeroides cells and eukaryotic cells such as plant, yeast, and other fungal cells. It is noted that cells containing an isolated nucleic acid of the invention are not required to express the isolated nucleic acid. In addition, the isolated nucleic acid can be integrated into the genome of the cell or maintained in an episomal state. In other words, cells can be stably or transiently transfected with an isolated nucleic acid of the invention.

Any method can be used to introduce an isolated nucleic acid into a cell. In fact, many methods for introducing nucleic acid into a cell, whether in vivo or in vitro, are well known to those skilled in the art. For example, calcium phosphate precipitation, conjugation, electroporation, heat shock, lipofection, microinjection, and viral-mediated nucleic acid transfer are common methods that can be used to introduce nucleic acid molecules into a cell. In addition, naked DNA can be delivered directly to cells in vivo as describe elsewhere (U.S. Pat. Nos. 5,580,859 and 5,589,466). Furthermore, nucleic acid can be introduced into cells by generating transgenic animals.

Any method can be used to identify cells that contain an isolated nucleic acid within the scope of the invention. For example, PCR and nucleic acid hybridization techniques such as Northern and Southern analysis can be used. In some cases, immunohistochemistry and biochemical techniques can be used to determine if a cell contains a particular nucleic acid by detecting the expression of a polypeptide encoded by that particular nucleic acid. For example, the polypeptide of interest can be detected with an antibody having specific binding affinity for that polypeptide, which indicates that that cell not only contains the introduced nucleic acid but also expresses the encoded polypeptide. Enzymatic activities of the polypeptide of interest also can be detected or an end product (e.g., a particular carotenoid) can be detected as an indication that the cell contains the introduced nucleic acid and expresses the encoded polypeptide from that introduced nucleic acid.

The cells described herein can contain a single copy, or multiple copies (e.g., about 5, 10, 20, 35, 50, 75, 100 or 150 copies), of a particular exogenous nucleic acid. All non-naturally-occurring nucleic acids are considered an exogenous nucleic acid once introduced into the cell. The term “exogenous” as used herein with reference to a nucleic acid and a particular cell refers to any nucleic acid that does not originate from that particular cell as found in nature. Nucleic acid that is naturally-occurring also can be exogenous to a particular cell. For example, an entire operon that is isolated from a bacteria is an exogenous nucleic acid with respect to a second bacteria once that operon is introduced into the second bacteria. For example, a bacterial cell (e.g., Rhodobacter) can contain about 50 copies of an exogenous nucleic acid of the invention. In addition, the cells described herein can contain more than one particular exogenous nucleic acid. For example, a bacterial cell can contain about 50 copies of exogenous nucleic acid X as well as about 75 copies of exogenous nucleic acid Y. In these cases, each different nucleic acid can encode a different polypeptide having its own unique enzymatic activity. For example, a bacterial cell can contain two different exogenous nucleic acids such that a high level of astaxanthin or other carotenoid is produced. In addition, a single exogenous nucleic acid can encode one or more polypeptides. For example, a single nucleic acid can contain sequences that encode three or more different polypeptides.

Microorganisms that are suitable for producing carotenoids may or may not naturally produce carotenoids, and include prokaryotic and eukaryotic microorganisms, such as bacteria, yeast, and fungi. In particular, yeast such as Phaffia rhodozyma (Xanthophyllomyces dendrorhous), Candida utilis, and Saccharomyces cerevisiae, fungi such as Neurospora crassa, Phycomyces blakesleeanus, Blakeslea trispora, and Aspergillus sp, Archaeabacteria such as Halobacterium salinarium, and Eubacteria including Pantoea species (formerly called Erwinia) such as Pantoea stewartii (e.g., ATCC Accession #8200), flavobacteria species such as Xanthobacter autotrophicus and Flavobacterium multivorum, Zymonomonas mobilis, Rhodobacter species such as R. sphaeroides and R. capsulatus, E. coli, and E. vulneris can be used. Other examples of bacteria that may be used include bacteria in the genus Sphingomonas and Gram negative bacteria in the α-subdivision, including, for example, Paracoccus, Azotobacter, Agrobacterium, and Erythrobacter. Eubacteria, and especially R. sphaeroides and R. capsulatus, are particularly useful. R. sphaeroides and R. capsulatus naturally produce certain carotenoids and grows on defined media. Such Rhodobacter species also are non-pyrogenic, minimizing health concerns about use in nutritional supplements. In some embodiments, it can be useful to produce carotenoids in plants and algae such as Zea mays, Brassica napus, Lycopersicon esculentum, Tagetes erecta, Haematococcus pluvialis, Dunaliella salina, Chlorella protothecoides, and Neospongiococcum excentrum.

It is noted that bacteria can be membranous or non-membranous bacteria. The term “membranous bacteria” as used herein refers to any naturally-occurring, genetically modified, or environmentally modified bacteria having an intracytoplasmic membrane. An intracytoplasmic membrane can be organized in a variety of ways including, without limitation, vesicles, tubules, thylakoid-like membrane sacs, and highly organized membrane stacks. Any method can be used to analyze bacteria for the presence of intracytoplasmic membranes including, without limitation, electron microscopy, light microscopy, and density gradients. See, e.g., Chory et al., (1984) J. Bacteriol., 159:540-554; Niederman and Gibson, Isolation and Physiochemical Properties of Membranes from Purple Photosynthetic Bacteria. In: The Photosynthetic Bacteria, Ed. By Roderick K. Clayton and William R. Sistrom, Plenum Press, pp. 79-118 (1978); and Lueking et al., (1978) J. Biol. Chem., 253: 451-457. Examples of membranous bacteria that can be used include, without limitation, Purple Non-Sulfur Bacteria, including bacteria of the Rhodospirillaceae family such as those in the genus Rhodobacter (e.g., R. sphaeroides and R. capsulatus), the genus Rhodospirillum, the genus Rhodopseudomonas, the genus Rhodomicrobium, and the genus Rhodophila. The term “non-membranous bacteria” refers to any bacteria lacking intracytoplasmic membrane. Membranous bacteria can be highly membranous bacteria. The term “highly membranous bacteria” as used herein refers to any bacterium having more intracytoplasmic membrane than R. sphaeroides (ATCC 17023) cells have after the R. sphaeroides (ATCC 17023) cells have been (1) cultured chemoheterotrophically under aerobic condition for four days, (2) cultured chemoheterotrophically under anaerobic for four hours, and (3) harvested. Aerobic culture conditions include culturing the cells in the dark at 30° C. in the presence of 25% oxygen. Anaerobic culture conditions include culturing the cells in the light at 30° C. in the presence of 2% oxygen. After the four hour anaerobic culturing step, the R. sphaeroides (ATCC 17023) cells are harvested by centrifugation and analyzed.

Nucleic acids of the invention can be expressed in microorganisms so that detectable amounts of carotenoids are produced. As used herein, “detectable” refers to the ability to detect the carotenoid and any esters or glycosides thereof using standard analytical methodology. In general, carotenoids can be extracted with an organic solvent such as acetone or methanol and detected by an absorption scan from 400-500 nm in the same organic solvent. In some cases, it is desirable to back-extract with a second organic solvent, such as hexane. The maximal absorbance of each carotenoid depends on the solvent that it is in. For example, in acetone, the maximal absorbance of lutein is at 451 nm, while maximal absorbance of zeaxanthin is at 454 mn. In hexane, the maximal absorbance of lutein and zeaxanthin is 446 nm and 450 nm, respectively. High performance liquid chromatography coupled to mass spectrometry also can be used to detect carotenoids. Two reverse phase columns that are connected in series can be used with a solvent gradient of water and acetone. The first column can be a C30 specialty column designed for carotenoid separation (e.g., YMCä Carotenoid S3m; 2.0×150 mm, 3 mm particle size; Waters Corporation, PN CT99S031502WT) followed by a C8 Xterraä MS column (e.g., Xterraä MS C8; 2.1×250 mm, 5 mm particle size; Waters Corporation, PN 186000459).

Detectable amounts of carotenoids include 10 μg/g dry cell weight (dcw) and greater. For example, about 10 to 100,000 μg/g dcw, about 100 to 60,000 μg/g dcw, about 500 to 30,000 μg/g dcw, about 1000 to 20,000 μg/g dcw, about 5,000 to 55,000 μg/g dcw, or about 30,000 μg/g dcw to about 55,000 μg/g dcw. With respect to algae or other plants or organisms that produce a particular carotenoid, such as astaxanthin, β-carotene, lycopene, or zeaxanthin, “detectable amount” of carotenoid is an amount that is detectable over the endogenous level in the plant or organism.

Depending on the microorganism and the metabolites present within the microorganism, one or more of the following enzymes may be expressed in the microorganism: geranylgeranyl pyrophosphate synthase, phytoene synthase, phytoene desaturase, lycopene βcyclase, lycopene εcyclase, zeaxanthin glycosyl transferase, β-carotene hydroxylase, β-carotene C-4 ketolase, and multifunctional geranylgeranyl pyrophosphate synthase. Suitable nucleic acids encoding these enzymes are described above. Also, see, for example, Genbank Accession No. Y15112 for the sequence of carotenoid biosynthesis genes of Paracoccus marcusii; Genbank Accession No. D58420 for the carotenoid biosynthesis genes of Agrobacterium aurantiacum; Genbank Accession No. M87280 M99707 for the sequence of carotenoid biosynthesis genes of Erwinia herbicola; and Genbank Accession No. U62808 for carotenoid biosynthesis genes of Flavobacterium sp. Strain R1534.

For example, to produce lycopene in a microorganism that naturally produces neurosporene, such as Rhodobacter, an exogenous nucleic acid encoding phytoene desaturase can be expressed, e.g., a phytoene desaturase of the invention, and lycopene can be detected using standard methodology. Expression of additional carotenoid genes in such an engineered cell will allow for production of additional carotenoids. For example, expression of a lycopene β-cyclase in such an engineered cell allows production of detectable amounts of β-carotene, while further expression of a β-carotene hydroxylase allows production of another carotenoid, zeaxanthin. β-carotene and zeaxanthin can be detected using standard methodology and are distinguished by mobility on an HPLC column. Zeaxanthin diglucoside can be produced by fuirther expression of zeaxanthin glucosyl transferase (crtX) in an organism that produces zeaxanthin.

Alternatively, canthaxanthin can be produced in organisms that produce phytoene by expression of phytoene desaturase, lycopene β-cyclase, and β-carotene C4 oxygenase, an enzyme that converts the methylene groups at the C4 and C4′ positions of the carotenoid to ketone groups. The β-carotene C4 oxygenase from, e.g., Agrobacterium aurantiacum or Haematococcus pluvialis can be used. See, GenBank Accession Nos. 1136630 and X86782 for a description of the nucleotide and amino acid sequences of the A. aurantiacum and H. pluvialis enzymes, respectively. The β-carotene C4 oxygenase from Brevundimonas aurantiaca also can be used. See, Example 2 for a description of the nucleotide and amino acid sequences. In organisms that do not naturally produce carotenoids, additional enzymes are required for production of canthaxanthin. Geranylgeranyl pyrophosphate synthase and phytoene synthase can be expressed such that the necessary precursors for canthaxanthin synthesis are present.

Astaxanthin also can be produced in microorganisms that naturally produce carotenoids. For example, a Rhodobacter cell can be engineered such that phytoene desaturase, lycopene β-cyclase, β-carotene hydroxylase, and β-carotene C4 oxygenase are expressed and detectable amounts of astaxanthin are produced. Such an organism also can express an enzyme that can modify the 3 or 3′hydroxyl groups of astaxanthin with chemical groups such as glucose (e.g., to produce astaxanthin diglucoside), other sugars, or fatty acids. In addition, a P. stewartii cell can be engineered such that β-carotene C4 oxygenase is expressed and detectable amounts of astaxanthin are produced. Astaxanthin can be detected as described above, and has maximal absorbance at 480 nm in acetone.

Yields of astaxanthin and other carotenoids can be increased by expression of a multifunctional geranylgeranyl pyrophosphate synthase, such as that from S. shibatae (SEQ ID NO:45) or an Archaebacterial gene from Archaeoglobus fulgidus (GenBank Accession No. AF120272), in the engineered microorganism. The archaebacteria GGPPS gene is a homolog of the endogenous Rhodobacter gene and encodes an enzyme that directly converts 3 IPP molecules and 1 DMAPP molecule to 1 GGPPS molecule, thereby reducing branching of the carotenoid pathway and eliminating production of other less desirable isoprenoids. Further reductions in less desirable metabolites can be obtained by eliminating endogenous bacteriochlorophyll biosynthesis, which redirects flow into carotenoid biosynthesis. For example, the bchO, bchD, and bchI genes can be deleted and/or replaced with an Archaebacterial GGPPS gene. Additional increases in yield can be obtained by deletion of the endogenous crtE gene or the endogenous crtC, crtD, crtE, crtA, crtI, and crtF genes. For example, the down regulation of the crtC gene was shown to increase neurosporene production in Rhodobacter sphaeroides (see Example 8 provided herein).

Carotenoid production in Rhodobacter also can be increased by down regulating and/or disruption of at least a portion of regulatory genes such as the ppsR, ccoN and/or aerR (also known as orf192, ppa, and ppsS genes) [Mol Microbiol. 2001 March; 39(5): 1116-23. Generalized approach to the regulation and integration of gene expression. Oh J I, Kaplan S]. The aerR gene is an aerobic repressor of photosynthesis gene expression and is located next to the ppsR gene on the Rhodobacter chromosome. Down regulation of the ppsR gene has previously been shown to increase carotenoid production [J Bacteriol. 2000 April; 182(8): 2253-61. Domain structure, oligomeric state, and mutational analysis of PpsR, the Rhodobacter sphaeroides repressor of photosystem gene expression. Gomelsky M, Home I M, Lee H J, Pemberton J M, McEwan A C; Kaplan S.]. Similarly, down regulation of the ccoN gene has previously been shown to increase photosynthesis gene expression in the presence of oxygen [Biochemistry. 1999 Mar. 2; 38(9):2688-96. The cbb3 terminal oxidase of Rhodobacter sphaeroides 2.4.1: structural and functional implications for the regulation of spectral complex formation. Oh J I, Kaplan S.], and the aerR gene has previously been shown to code for an aerobic repressor of photosynthesis gene expression in Rhodobacter capsulatus [J Bacteriol. 2002 May;184(10):2805-14. AerR, a second aerobic repressor of photosynthesis gene expression in Rhodobacter capsulatus. Dong C, Elsen S, Swem L R, Bauer C E.]. In other embodiments, a microorganism can include a genomic disruption of at least a portion of a ppsR nucleic acid sequence and at least a portion of an aerR nucleic acid sequence such that the ppsR and aerR nucleic acid sequences are non-functional. Given this knowledge it is predictable that a combination of down regulating one or more of the ppsR, ccoN, and/or aerR genes individually in combination with the down regulation of the crtC gene or in combinations combined with the down regulation of the crtC gene will enhance carotenoid production.

More specifically, the nucleic acid sequence of the ccoN gene from R. sphaeroides and R. capsulatus can be found in GenBank (Accession Nos. U58092 and AF016223, respectively). The ppsR gene encodes a transcription factor that represses carotenoid and bacteriochlorophyll synthesis under both aerobic and anaerobic conditions. The nucleic acid sequence of the ppsR gene from R. sphaeroides can be found in GenBank (Accession No. L37197). The R. capsulatus homolog of ppsR is called crtJ, the nucleic acid sequence of which can be found in GenBank under Accession No. Z 11165.

Common mutagenesis or knock-out technology can be used to delete endogenous genes. Alternatively, antisense technology can be used to reduce enzymatic activity. For example, a R. sphaeroides cell can be engineered to contain a cDNA that encodes an antisense molecule that prevents an enzyme from being made. The term “antisense molecule” as used herein encompasses any nucleic acid that contains sequences that correspond to the coding strand of an endogenous polypeptide. An antisense molecule also can have flanking sequences (e.g., regulatory sequences). Thus, antisense molecules can be ribozymes or antisense oligonucleotides. A ribozyme can have any general structure including, without limitation, hairpin, hammerhead, or axhead structures, provided the molecule cleaves RNA.

Control of the Ratio of Carotenoids

The amount of particular carotenoids, such as astaxanthin to canthaxanthin, or astaxanthin to zeaxanthin, can be controlled by expression of carotenoid genes from an inducible promoter or by use of constitutive promoters of different strengths. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by heat, cold, salt, or toxic elements, or indirectly through the action of a pathogen or disease agent such as a virus. The inducer also can be an illumination agent such as light, darkness and light's various aspects, which include wavelength, intensity, fluorescence, direction, and duration. Examples of inducible promoters include the lac system and the tetracycline resistance system from E. coli. In one version of the lac system, expression of lac operator-linked sequences is constitutively activated by a lacR-VP 16 fusion protein and is turned off in the presence of IPTG. In another version of the lac system, a lacR-VP 16 variant is used that binds to lac operators in the presence of IPTG, which can be enhanced by increasing the temperature of the cells.

Components of the tetracycline (Tc) resistance system also can be used to regulate gene expression. For example, the Tet repressor (TetR), which binds to tet operator sequences in the absence of tetracycline and represses gene transcription, can be used to repress transcription from a promoter containing tet operator sequences. TetR also can be fused to the activation domain of VP 16 to create a tetracycline-controlled transcriptional activator (tTA), which is regulated by tetracycline in the same manner as TetR, i.e., tTA binds to tet operator sequences in the absence of tetracycline but not in the presence of tetracycline. Thus, in this system, in the continuous presence of Tc, gene expression is repressed, and to induce transcription, Tc is removed.

Alternative methods of controlling the ratio of carotenoids include using enzyme inhibitors to regulate the activity levels of particular enzymes.

Production of Carotenoids

Carotenoids can be produced in vitro or in vivo. For example, one or more polypeptides of the invention can be contacted with an appropriate substrate or combination of substrates to produce the desired carotenoid (e.g., astaxanthin). See, FIG. 1 for a schematic of the carotenoid biosynthetic pathway.

A particular carotenoid (e.g., astaxanthin, lycopene, β-carotene, lutein, zeaxanthin, zeaxanthin diglucoside, or canthaxanthin) also can be produced by providing an engineered microorganism and culturing the provided microorganism with culture medium such that the carotenoid is produced. In general, the culture media and/or culture conditions are such that the microorganisms grow to an adequate density and produce the desired compound efficiently. For large-scale production processes, the following methods can be used. First, a large tank (e.g., a 100 gallon, 200 gallon, 500 gallon, or more tank) containing appropriate culture medium with, for example, a glucose carbon source is inoculated with a particular microorganism. After inoculation, the microorganisms are incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank. For example, the first tank can contain medium with xylose, while the second tank contains medium with glucose.

Once transferred, the microorganisms can be incubated to allow for the production of the desired carotenoid. Once produced, any method can be used to isolate the desired compound. For example, if the microorganism releases the desired carotenoid into the broth, then common separation techniques can be used to remove the biomass from the broth, and common isolation procedures (e.g., extraction, distillation, and ion-exchange procedures) can be used to obtain the carotenoid from the microorganism-free broth. In addition, the desired carotenoid can be isolated while it is being produced, or it can be isolated from the broth after the product production phase has been terminated. If the microorganism retains the desired carotenoid, the biomass can be collected and the carotenoid can be released by treating the biomass or the carotenoid can be extracted directly from the biomass. Extracted carotenoid can be formulated as a nutraceutical. As used herein, a nutraceutical refers to a compound(s) that can be incorporated into a food, tablet, powder, or other medicinal form that, upon ingestion by a subject, provides a specific medical or physiological benefit to the subject.

Alternatively, the biomass can be collected and dried, without extracting the carotenoids. The biomass then can be formulated for human consumption (e.g., as a dietary supplement) or as an animal feed (e.g., for companion animals such as dogs, cats, and horses, or for production animals). For example, the biomass can be formulated for consumption by poultry such as chickens and turkeys, or by cattle, pigs, and sheep. Feeding of such compositions may increase yield of breast meat in poultry and may increase weight gain in other farm animals. In addition, the carotenoids may increase shelf-life of meat products due to the increased antioxidant protection afforded by the carotenoids. The biomass also can be formulated for use in aquaculture. For example, biomass that includes an engineered microorganism that is producing, e.g., astaxanthin and/or canthaxanthin, can be fed to fish or crustaceans to pigment the flesh or carapace, respectively. Such a composition is particularly useful for feeding to fish such as salmon, trout, sea breem, or snapper, or crustaceans such as shrimp, lobster, and crab.

One or more components can be added to the biomass before or after drying, including vitamins, other carotenoids, antioxidants such as ethoxyquin, vitamin E, butylated hydroxyanisole (BHA), butylated hydroxytoluene (BHT), or ascorbyl palmitate, vegetable oils such as corn oil, safflower oil, sunflower oil, or soybean oil, and an edible emulsifier, such as soy bean lecithin or sorbitan esters. Addition of antioxidants and vegetable oils can help prevent degradation of the carotenoid during processing (e.g., drying), shipment, and storage of the composition.

The invention will be further described in the following examples, which do not limit the scope of the invention described in the claims.

### EXAMPLES

### Example 1

### Cloning of the Zeaxanthin Gene Cluster from Pantoea stewartii

Genomic DNA from P. stewartii was isolated and digested with restriction enzymes to yield genomic DNA fragments approximately 8-10 kB in size. These genomic DNA fragments were ligated into a vector cut with the same restriction enzyme, and electroporated into electrocompetent E. coli. Transformant colonies were individually picked and transferred onto fresh solid media with the appropriate antibiotic selection (ampicillin/ampicillin substitute). It was thought that E. coli colonies containing the P. stewartii carotenoid genes would appear yellow in color due to the production of zeaxanthin pigment or red due to the production of lycopene. Although at least 2000 ampicillin resistant E. coli transformants were screened, none of the colonies were found to contain the P. stewartii carotenoid genes.

Instead, a second, PCR based method was used to identify and sequence the carotenoid (crt) gene cluster from P. stewartii genomic DNA. Degenerate primers were designed based on homologous regions identified in the crt genes from Erwinia herbicola and Erwinia uredovora. Table 2 provides the position of the crt genes in E. herbicola and E. uredovora.

TABLE 2
Position of crt genes in E. herbicola and E. uredovora

| | Start of Gene | | End of Gene | |
|---|---|---|---|---|
| Gene | (nucleotide #) | | (nucleotide #) | |
| name | E. herbicola | E. uredovora | E. herbicola | E. uredovora |
| CrtE | 3535 | 198 | 4458 | 1133 |
| Orf-6 | 4521 | | 5564 | |
| CrtX | 5561 | 1143 | 6802 | 2438 |
| CrtY | 6799 | 2422 | 7959 | 3570 |
| CrtI | 7956 | 3582 | 9434 | 5060 |
| CrtB | 9431 | 5096 | 10360 | 5986 |
| CrtZ | 10826 | 6452 | 10296 | 5925 |
| | (complement) | (complement) | complement | (complement) |
| Orf-12 | 12127 | | 10916 | |
| | complement | | complement | |

The following primers were designed (Table 3) and used in various combinations to yield PCR products of varying lengths. P. stewartii genomic DNA was used as template.

TABLE 3
Sequences of Degenerate Primers

| | | |
|---|---|---|
| | | SEQ ID |
| Primer Name | Primer Sequence | NO |

| | | |
|---|---|---|
| P.s.BCHy1 | 5′-ATYATGCACGGCTGGGGWTGGSGMTGG | 13 |
| | CA-3′ | |
| P.s.BCHy2 | 5′-GGCCARCGYTGATGCACCAGMCCGTCRTG | 14 |
| | CA-3′ | |
| P.s.PS1 | 5′-CTGATGCTCTAYGCCTGGTGCCGCCA-3′ | 15 |
| P.s.PS2 | 5′-TCGCGRGCRATRTTSGTCARCTG-3′ | 16 |
| P.s.LBC1 | 5′-ATBMTSATGGAYGCSACSGT-3′ | 17 |
| P.s.LBC2 | 5′-YTRATCGARGAYACGCRCTA-3′ | 18 |
| P.s.LBC3 | 5′-RSGGCAGYGAATAGCCRGTG-3′ | 19 |
| P.s.LBC4 | 5′-AACAGCATSCGRTTCAGCAKGCGSA-3′ | 20 |
| P.s.PD5 | 5′-CCGACGGTKATCACCGATCC-3′ | 21 |
| P.s.PD6 | 5′-CTGCGCCSACCAGGTAGAG-3′ | 22 |
| P.sGGPPS1 | 5′-CTYGACGAYATGCCCTGCATGGAC-3′ | 23 |
| | (MD92) | |
| P.s.GGPPS2 | 5′-GTCGATTTWCCSGCGTCCTKATTG-3′ | 24 |
| | (MD93) | |

PCR was performed in a Gradient Thermocycler, and was started by incubating at 96° C. for 5 minutes, followed by 40 cycles of denaturation at 96° C. for 30 seconds, annealing at 40° C./45° C./50° C./55° C./or 60° C. for 105 seconds, and extension 90 seconds, followed by incubation at 72° C. for 10 mins. The concentration of MgCl₂ in the PCR reactions also was varied and ranged from a final concentration of 1.5 mM to 6 mM. Table 4 provides the predicted size of the PCR products with various primer combinations.

TABLE 4
Expected sizes of PCR Products

| Primer Combination | PCR product length (bp) | Product Observed |
|---|---|---|
| BCHy1/BCHy2 | 230 | Yes |
| PS1/PS1 | 410 | Yes |
| LBC1/LBC3 | 320 | Yes |
| LBC1/LBC4 | 460 | Yes |
| PD1/PD2 | 420 | No |
| PD1/PD4 | 1260 | No |
| LBC2/LBC3 | 240 | No |
| PD3/PD4 | 410 | Yes |
| LBC2/LBC4 | 380 | Yes |
| PD5/PD6 | 1200 | Yes |
| PS1/PS2 | 410 | Yes |
| BCHy1/BCHy2 | 230 | Yes |
| PsGGPPS1/PsGGPPS2 | 470 | Yes |
| LBCDown1/PDUp1 | 470 | Yes |
| PDDown1/PSUp1 | 300 | Yes |
| BCHyDown1/PSDown1 | 700 | Yes |
| LBCUp1/GGPPSdn1 | 1600 | Yes |

PCR reactions were electrophoresed through agarose gels to estimate sizes of PCR products and DNA was extracted from the gel using a Qiagen gel extraction kit. The purified PCR products were submitted to the Advanced Genetic Analysis Center (AGAC) at the University of Minnesota for sequencing. The obtained DNA sequences were subjected to BLAST analysis to determine if the sequences were homologous to crt genes from other bacteria. Sequence analysis of the 1.2-kb DNA fragment indicated that there was homology to phytoene desaturase (crt1) genes from E. herbicola and E. uredovora, while the 0.47 kB product had homology with the crtE genes from E. herbicola and E. uredovora.

Based on the DNA sequence information generated using the degenerate primers and amplified regions of the carotenoid genes from P. stewartii, primers specific for the P. stewartii crt genes were designed and are shown in Table 5. These specific primers were used to obtain information upstream and downstream of the DNA regions amplified with the degenerate primers. This rationale was used to extend and obtain DNA sequence information about the P. stewartii crt genes.

TABLE 5
P. stewartii primers

| | | |
|---|---|---|
| | | SEQ |
| | | ID |
| Primer | Sequence | NO |

| | | |
|---|---|---|
| PsOp.crtE | 5′-GGCCGAATTCCAACGATGCTCTGGCA | 25 |
| | GTTA-3′ | |
| PSOp.crtZ(−) | 5′-GGCCAGATCTACTTCAGGCGACGCTGA | 26 |
| | GAG-3′ | |
| PsOp.crtZ(+) | 5′-GGCCAGATCTTACGCGCGGGTAAAGCC | 27 |
| | AAT-3′ | |
| PsOp.crtZ(2+) | 5′-GGCCTCTAGAATTACCGCGTGGTTCTG | 28 |
| | AAG-3′ | |
| PsOp.crtZ(2−) | 5′-GGCCTCTAGATCTGTACGCGCCACCGT | 29 |
| | TAT-3′ | |

After unsuccessful attempts at completing the sequence crt gene cluster sequence from P. stewartii using PCR, the Universal Genome Walker kit from Clontech was used to obtain the complete the sequence of the P. stewartii crtE and crtZ genes. This kit uses a PCR based approach. The following primer pairs were synthesized and used for the genome walking experiments: GWcrtE2, 5′-CATCGGTAAGATCGTCAAGCAACTGAA-3′ (SEQ ID NO:30) and GWcrtE1, 5′-GATTTACCTGCATCCTGATTGATGTCT-3′ (SEQ ID NO:31); and GWcrtZ1, 5′-ATGTATAACCGTTTCAGGTAGCCTTTG-3′ (SEQ ID NO:32) and GWcrtZ2, 5′-AATACAGTAAACCATAAGCGGTCATGC-3′ (SEQ ID NO:33). The sequences of the crt genes and encoded proteins from P. stewartii were compared to the sequence of the crt genes and proteins from E. herbicola and E. uredovora using BLAST under default parameters. See, SEQ ID NOS 1-12 for the nucleotide and amino acid sequences of the P. stewartii crt genes. The results of the alignment are provided in Table 6.

TABLE 6
Comparison of crt genes and proteins from P. stewartii
to E. herbicola and E. uredovora

| | Comparison | | Comparison | |
|---|---|---|---|---|
| | of nucleotide | | of protein | |
| | sequence of | | sequence of | |
| | P. stewartii to | | P. stewartii to | |
| Gene | E. herbicola | E. uredovora | E. herbicola | E. uredovora |
| crtE | 59% | 80% | 81% | 83% |
| crtX | 56% | 75% | 75% | 74% |
| crtY | 58% | 77% | 83% | 82% |
| crtI | 69% | 81% | 89% | 89% |
| crtB | 63% | 81% | 88% | 88% |
| crtZ | 65% | 84% | 65% | 88% |

### Example 2

Cloning of a β-carotene C4 oxygenase from Brevundimonas aurantiaca: Degenerate PCR primers for crtW were designed based on crtW genes from Bradyrhizobium, Alcaligenes, Agrobacterium aurantiacum, and Paracoccus marcusii. The primers had the following sequences: (crtW(181P.m.)-5′TTCATCATCGCGCATGAC3′ (SEQ ID NO:34) and crtW(668P.m.)-5′AGRTGRTGYTCGTGRTGA (SEQ ID NO:35), and were synthesized by Integrated DNA Technologies Inc. (Coralville, Iowa). PCR was performed in a mastercycler gradient machine (Eppendorf) with genomic DNA from B. aurantiaca (ATCC Accession No. 15266). Reaction conditions included five minutes at 96° C., followed by 30 cycles of denaturation at 94° C. for 30 sec., annealing at 50° C. for 2 min., and extension at 72° C. for 2 min 30 sec, and a final 72° C. incubation for 10 min. An approximately 500-bp PCR product was obtained and cloned into the vector pCR-BluntII-TOPO (Invitrogen Corp. Carlsbad, Calif.).

Independent clones were sequenced using the universal M13 forward and reverse primers. DNA sequencing was carried out at AGAC, University of Minnesota, St. Paul, Minn. Partial nucleotide sequence of the crtW gene was obtained. Alignment of the partial sequence with known crtW genes indicated that the sequences aligned toward the N-terminus and C-terminus, respectively, of the crtW genes from Bradyrhizobium, Alcaligenes, Agrobacterium aurantiacum, and Paracoccus marcusii. The Universal Genome Walker kit from Clontech was used to obtain the complete the sequence of the B. aurantiaca crtW gene. Primers were synthesized based on the partial sequence and used for the genome walking experiments.

Upon obtaining sequence from the ends of the gene, the following oligonucleotide primers were synthesized and used to amplify the complete crtW gene from genomic DNA: 5′-GCGGCATAGGCTAGATTGAAG-3′ (primer 1, Tm=72° C., SEQ ID NO:36) and 5′-GCGAGTTCCTTCTCACCTAT-3′ (primer 2, Tm=67° C., SEQ ID NO:37). B. aurantiaca (ATCC 15266) genomic DNA was prepared with the Qiagen genomic-tip 500G kit (Valencia, Calif.; Catalog #10262) following the manufacturers protocol. Briefly, 30 ml of B. aurantiaca culture were grown overnight at 30° C. in ATCC medium 36 (Caulobacter medium; 2 g/l peptone, 1 g/l yeast extract, 0.2 g/l MgSO4.7H20). Cultures were harvested by centrifuigation (15,000×g; 10 minutes) and genomic DNA purified following the manufacturer's recommended protocol (Qiagen Genomic DNA Handbook for Blood, Cultured Cells, Tissue, Mouse Tails, Yeast, Bacteria (Gram- & some Gram+). The Expand DNA polymerase system (Roche Molecular Biochemicals, Indianapolis, Ind.; catalog #1732641) was used in a reaction that included 2 μl of B. aurantiaca genomic DNA (50 ng/μl), 1 μl of primer 1 (100 pmol/μl), 1 μl of primer 2 (100 pmol/μl), 5 μl of 10×PCR buffer, 1 μl of Expand DNA polymerase (3.5 U/μl), 2.5 μl of dimethyl sulfoxide (DMSO), 2 μl of dNTP's (10 nmol/μl each), and 35.5 μl of dd H₂O. Reaction conditions included five minutes at 96° C., followed by 30 cycles of denaturation at 94° C. for 30 sec., annealing at 50° C. for 2 min., and extension at 72° C. for 2 min 30 sec, and a final 72° C. incubation for 10 min.

PCR products were electrophoresed through a 0.8% agarose gel and the ˜0.85 kB band was excised from the gel and purified using the Qiagen QIAquick Gel Extraction Kit (catalog #28704) following the manufacturer's recommended protocol (QIAquick Spin Handbook). Gel-purified PCR product was cloned into the blunt-end cloning site of pCR-Blunt II-TOPO (Clontech; Palo Alto, Calif.) to generate pTOPOcrtW. Ligation mixtures were electroporated (25 μF, 200 Ohms, 12.5 KV/cm) into E. coli DH10B electromax cells (Gibco BRL; Gaithersburg, Md.; catalog #18290-015). Transformants were allowed to recover 60 minutes at 37° C. with shaking in 1 ml of SOC medium. Cells were plated on LB agar +50 μg/ml kanamycin and allowed to grow overnight at 37° C. Transformant colonies were inoculated into 1 ml LB broth +50 μg/ml kanamycin and allowed to grow overnight at 37° C. with shaking. Minipreps were prepared using the QIAprep Spin Miniprep Kit (50) (catalog #27104) following the manufacturer's protocol and the presence of pTOPOcrtW was screened for by restriction analysis with EcoRI. EcoRI digests of pTOPOcrtW yielded products of ˜0.85 Kbp and 3.5 Kbp.

The crtW gene was sequenced by AGAC, University of Minnesota, St. Paul, Minn. The nucleotide sequence of the crtW gene from B. aurantiaca is provided in SEQ ID NO:38, and the protein encoded by the crtW gene is provided in SEQ ID NO:39.

### Example 3

Transformation of pTOPOcrtW into Pantoea stewartii and production of astaxanthin and adonixanthin in P.stewardii::pTOPOcrtW: The following protocol describes expression of crtW in the zeaxanthin producing host P. stewartii. This yields a transformed host that is capable of producing astaxanthin (i.e., 3,3′-dihydroxy-β,β-carotene-4,4′-dione) and adonixanthin (3,3′-dihydroxy-β,β-carotene-4-one). Electrocompetent P. stewartii (ATCC 8200) cells were prepared by culturing 50 ml of a 5% inoculum of P. stewartii cells in LB at 30° C.—with agitation (250 rpm) until an OD₅₉₀ of 0.5-1.0 was reached. The bacteria were washed in 50 ml of 10 mM HEPES (pH 7.0) and centrifuged for 10 minutes at 10,000×g. The wash was repeated with 25 ml of 10 mM HEPES (pH 7.0) followed by the same centrifugation protocol. The cells then were washed once in 25 ml of 10% glycerol. Following centrifugation, the cells were resuspended in 500 μl of 10% glycerol. Forty μl aliquots were frozen and kept at −80° C. until use.

Plasmid TOPOcrtW was electroporated into electrocompetent P. stewartii cells (25 μF, 25 KV/cm, 200 Ohms) and plated onto LB agar plates containing 50 μg/ml kanamycin. As a negative control, pCR-Blunt II-TOPO self-ligated parental vector also was electroporated into P. stewartii and plated onto LB agar plates containing 50 μg/ml kanamycin. Individual colonies of P. stewartii::pTOPOcrtW were screened by visual inspection for a phenotypic change from bright yellow pigmentation (production of zeaxanthin) to a reddish-orange pigmentation (production of astaxanthin) and chosen for further pigment analysis. No phenotypic change was noted for individual colonies of P. stewartii::pCR-Blunt II-TOPO, so clones were randomly chosen for pigment analysis.

Production of astaxanthin was confirmed by HPLC/MS. Carotenoids were extracted from cells harvested from 5 day old cultures of P. stewartii::pTOPOcrtW or P. stewartii:: pCR-Blunt II-TOPO (25 ml) grown in LB with 50 μg/ml kanamycin by resuspending the washed cell pellet in 5 ml of acetone. Glass beads were added and the mixture was incubated for 60 minutes at room temperature in the dark with occasional vortexing. The cells were separated from the acetone extract by centrifugation at 15,000×g for 10 minutes. The acetone supernatant then was analyzed by HPLC/MS.

A Waters 2790 LC system was used with two reverse-phase C30 specialty columns designed for carotenoid separation (YMCa Carotenoid S3m; 2.0×150 mm, 3 mm particle size; Waters Corporation, PN CT99S031502WT)), in tandem. The columns were run at room temperature. A gradient of Mobile Phase A (0.1% acetic acid) and Mobile Phase B (90% acetone) was used to separate zeaxanthin and astaxanthin according to the following gradient timetable: 0 min (10% A, 90% B), 10 min (100% B), 12 min (10% A, 90% B), 15 min (10% A, 90% B). Flow rate was 0.3 ml/min. Samples were stored at 20° C. in an autosampler and a volume of 25 μL was injected. A Waters 996 Photodiode array detector, 350-550 nm, was used to detect zeaxanthin and astaxanthin. Under these chromatography conditions astaxanthin eluted at approximately 5.42-5.51 min and zeaxanthin eluted at approximately 6.22-6.4 min.

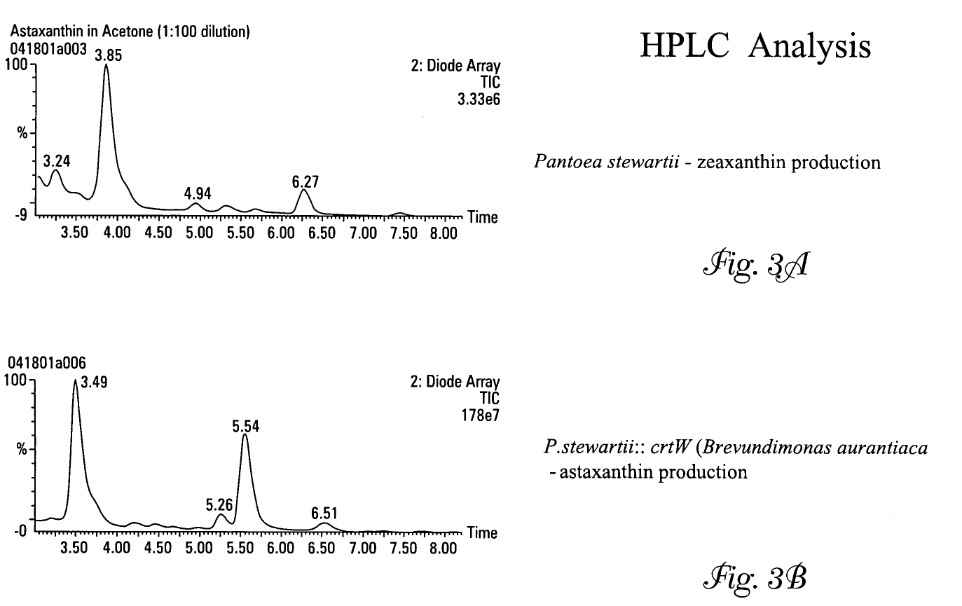

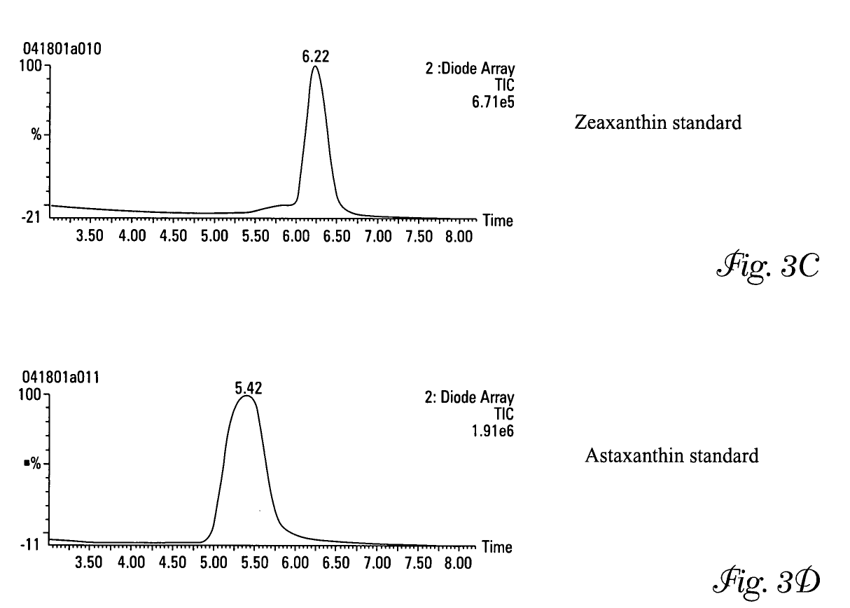

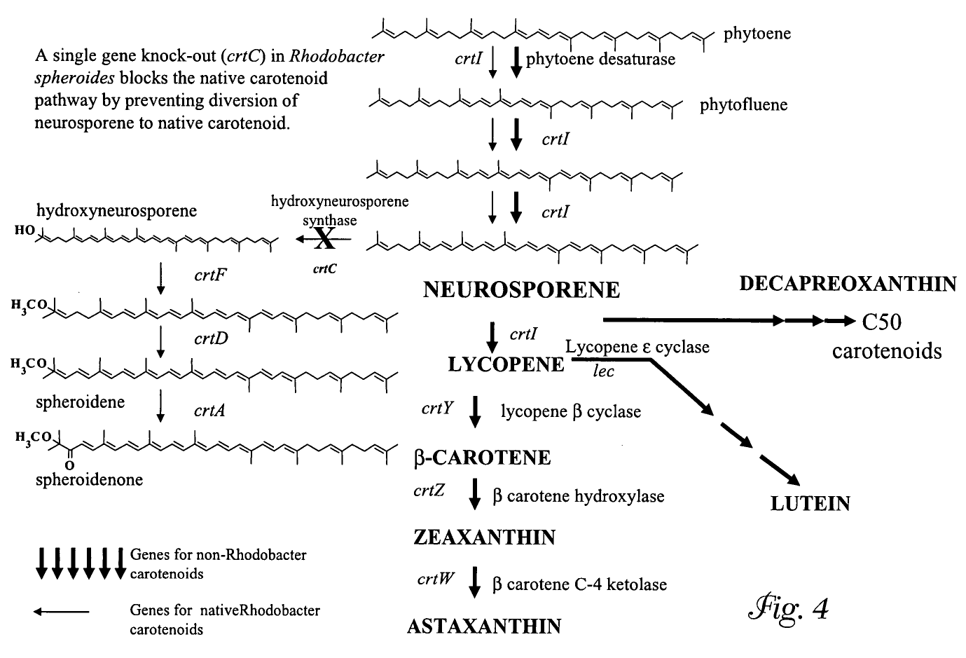

Carotenoid standards were used to identify the peaks. Astaxanthin was obtained from Sigma Chemical Co. (St. Louis, Mo.) and zeaxanthin was obtained from Extrasynthese (France). UV-Vis absorbtion spectra were used as diagnostic features for the carotenoids as were the molecular ion and fragmentation patterns generated using mass spectrometry. A positive-ion atmospheric pressure chemical ionization mass spectrometer was used; scan range, 400-800 m/z with a quadripole ion trap. A representative HPLC chromatogram is shown in FIG. 3, which confirms production of astaxanthin in P. stewartii transformed with the B. aurantiaca crtW gene.

### Example 4

Simultaneous Production of CoQ-10 and (3S, 3′S) Astaxanthin in a Microorganism: Although Phaffia rhodozyma is not capable of producing the 3S, 3′S isoform of astaxanthin, it is known to produce Coenzyme Q-10. This compound has been found to have particularly high value as a nutraceutical. The current invention is of particular value since R. sphaeroides is known to produce Coenzyme Q-10 and has been transformed with genes that, while novel, are nevertheless homologous to native genes in the MABP. Consequently, the described organism can be expected to simultaneously produce both Coenzyme Q-10 and (3S, 3′S)-ATX. This is the first described production of the production of both (3S, 3′S)-ATX and Coenzyme Q-10 in a single microbial host.

The identification of (3S, 3′S)-ATX can be accomplished as described by Maoka, T., et al. J. Chromatogr. 318:122-124 (1985). Briefly, this consists of extraction of the carotenoid pigments by contacting the biomass with a suitable organic solvent such as actetone or dichloromethane. The carotenoid extract is then dried under a stream of liquid nitrogen and resuspended in a solvent of n-hexane-dichloromethane-ethanol (48:16:0.6). The extract is applied to a Sumipax OA-2000 (particle size 10 uM) 250×4 mm I.D. (Sumitomo Chemicals, Osaka, Japan) chiral resolution HPLC column at a flow rate of 0.8 ml/min. Generally, the order of elution is expected to be (3R, 3′R)-ATX followed by (3R, 3′S; 3S, 3′R)-ATX followed by (3S, 3′S)-ATX. A similar separation is described in Maoka, T., et al. Comp. Biochem. Physiol. 83B:121-124 (1986). Briefly, this consists of isolation of the carotenoid, derivitization to the dibenzoate form with benzoyl chloride and separation of the enantiomers using a Sumipax OA-2000 chiral resolution HPLC column.

### Example 5

Transformation of the multifunctional GGPP synthase from Archeoglobus fulvidus into Rhodobacter strain ppsr- with the crtY and crtI genes from Pantoea stewartii inserted into the chromosome: The following protocol describes the generation of a β-carotene producing strain of R. sphaeroides (ATCC 35053), a facultative photoheterotroph, in which the ppsr gene was deleted by using the in-frame deletion procedure of Higuchi, R., et al, Nucleic Acid Res. 16: 7351-7367 to generate strain AREG. Table 7 describes the strains and plasmids used in this example. PpsR is a transcription factor that is involved in the repression of photosysem gene expression under aerobic growth conditions. The region of the chromosome that included the native tspO, crtC, crtD, crtE and crtF genes of AREG were replaced by the lycopene βcyclase (crtY) and phytoene desaturase (crtI) genes from P. stewartii using the procedure of Oh and Kaplan, Biochemistry 38:2688-2696 (1999); and Lenz, et al., J. Bacteriology 176:4385-4393 (1994), to generate the strain ΔREG(Δ5:YI). Briefly, the crtY and crt I genes were cloned into pLO1, a suicide vector for R. sphaeroides containing the Kanamycin resistance gene and the Bacillus subtilis sacB gene encoding sensitivity to sucrose. DNA fragments flanking the crtYI genes and identical in sequence to ˜500 bp internal fragments of the R. sphaeroides tspO and crtF genes were then cloned into pLO1. These flanking DNA regions correspond to the desired region for insertion of the crtYI genes. Insertion of the crtYI genes in AREG was confirmed using PCR analyses and appropriate PCR primers specific to the crtYI genes as well as flanking regions of the R.sphaeroides genome. The crtYI (P. stewartii) insertion and tspO, crtC, crtD, crtE and crtF (R. sphaeroides) deletion resulted in the lack of native carotenoid production and a change in the pigmentation from red to green, confirming the insertion event.

TABLE 7
Description of Rhodobacter Strains and Plasmids

| | | Major | |
|---|---|---|---|
| | | Carotenoid | |
| Strain | Description | Produced | Comments |
| ΔREG | ATCC 35053; | Sphaeroidenone | Regulatory |
| | ppsR regulatory mutant | (Native | mutant |
| | | Carotenoid) | |
| ΔREG(Δ5:YI) | CrtY and crtI genes of P. | None | β-carotene |
| | stewartii replaced 5 host | | biosynthetic |
| | genes (tspO, crtC, crtD, | | genes placed in |
| | crtE and crtF) on | | chromosome. No |
| | chromosome | | carotenoid |
| | | | production |
| | | | because of crtE |
| | | | deletion |
| ΔREG(Δ5:YI)::pP | Control vector introduced | None | Control vector |
| ctrl | into ΔREG(Δ5:YI) host | | contains rrnB |
| | | | promoter but no |
| | | | biosynthetic |
| | | | genes |
| ΔREG(Δ5:YI)::pP | gps gene of A. fulgidus | β-Carotene | gps gene on |
| gps | inserted into pPctrl control | | plasmid |
| | vector and introduced into | | complements crtE |
| | ΔREG(Δ5:YI) host | | deletion. |
| | | | Complete |
| | | | pathway for β- |
| | | | carotene |
| | | | production |
| ΔREG(Δ5:YI) | gps gene of A. fulgidus | β-Carotene | gps gene inserted |
| (ΔA:gps) | replaced crtA host gene on | | into genome |
| | chromosome of | | complements crtE |
| | ΔREG(Δ5:YI) host | | deletion. |
| | | | Complete |
| | | | pathway for β- |
| | | | carotene |
| | | | production |
| ΔREG(Δ5:YI) | crtW and crtZ genes | Astaxanthin | crtW and crtZ |
| (ΔA:gps) | inserted into pPctrl control | | genes convert β- |
| ::pPWZ | vector and introduced into | | carotene into |
| | ΔREG(Δ5:YI) (ΔA:gps) | | astaxanthin |
| | host | | |
| ΔREG(Δ5:YI) | gps, crtW and crtZ genes | Astaxanthin | Additional copies |
| (ΔA:gps) | inserted into pPctrl control | | of A. fulgidus gps |
| ::pPgpsWZ | vector and introduced into | | gene on plasmid |
| | ΔREG(Δ5:YI) (ΔA:gps) | | increases |
| | host | | production of |
| | | | astaxanthin |

| Plasmids | Genetic elements inserted |
|---|---|
| PBBR1MCS2 | None |
| PPctrl | rrnB promoter |
| PPgps | rrnB promoter, A. fulgidus |
| | gps |
| PPWZ | rrnB promoter, P. stewartii |
| | crtZ, |
| | B. aurantiacum crtW |
| PPgpsWZ | rrnB promoter, A. fulgidus |
| | gps |
| | P. stewartii crtZ, |
| | B. aurantiacum crtW |

The pPctrl vector was constructed by inserting a copy of the R. sphaeroides rrnB promoter (GenBank Accession #X53854; rrnBP) into the vector pBBRIMCS2 (GenBank Accession #U23751). The rrnB promoter was isolated from the vector pTEX24 (S. Kaplan) by a BamHI restriction enzyme digest, which released the promoter as a 363 bp fragment. This fragment was gel purified from a 2% Tris-acetate-EDTA (TAE) agarose gel. To prepare the pBBRIMCS2 vector for ligation, it also was digested with BamHI and the enzyme heat inactivated at 80° C. for 20 minutes. The digested vector was dephosphorylated with shrimp alkaline phosphatase (Roche Molecular Biochemicals, Indianapolis, Ind.), and gel purified from a 1% TAE-agarose gel. The prepared vector and the rrnB fragment were ligated using T4 DNA ligase at 16° C. for 16 hours to generate the plasmid pPctrl. One μL of ligation reaction was used to electroporate 40 μL of E. coli ElectromaxTM DH1OBTM cells (Life Technologies, Inc., Rockville, Md.).

Electroporated cells were plated on LB media containing 25 μg/mL of kanamycin (LBK). pPctrl DNA was isolated from cultures of single colonies and was digested with Hind III to confirm the presence of a single insertion of the rrnB promoter. The sequence of pPctrl also was confirmed by DNA sequencing.

The multifunctional GGPP synthase (gps) gene from A. fulgidus (GenBank Accession No. AF 120272) was cloned into the multiple cloning site of pPctrl to generate the construct pPgps.

Electrocompetent ΔREG(A5:YI) cells were prepared as follows: 5 ml cultures were inoculated using Sistrom's media supplemented with trace elements, vitamins (O'Gara, et al., J. Bacteriol. 180:4044-4050 (1988); Cohen-Bazire, et al. J. Cell. Comp. Physiol. 49:25-68 (1957)) and 0.4% glucose as a carbon source, and grown overnight at 30° C. with shaking. This culture was diluted 1/100 in 300 mL of the same media and grown to an OD₆₆₀ of 0.5-0.8. The cells were chilled on ice for 10 minutes and then centrifuged for 6 minutes at 7,500 g. The supernatant was discarded and the cell pellet was resuspended in ice-cold 10% glycerol at half of the original volume. The cells were pelleted by centrifugation for 6 minutes at 7,500 g. The supernatant was again discarded and cells were resuspended in ice cold 10% glycerol at one quarter of the original volume. The last centrifugation and resuspension steps were repeated, followed by centrifugation for 6 minutes at 7,500 g. The supernatant was decanted and the cells resuspended in the small volume of glycerol that did not drain out. Additional ice-cold 10% glycerol was added to resuspend the cells if necessary. Forty μL of the resuspended cells was used in a test electroporation (see below) to determine if the cells needed to be concentrated by centrifugation or diluted with 10% ice-cold glycerol. Time constants of 8.5-9.0 resulted in good transformation efficiencies. Once an acceptable time constant was achieved, cells were aliquoted into cold microfuge tubes and stored at −80° C. All water used for media and glycerol was 18 Mohm or higher.

Electroporation of ΔREG(A5:YI) was carried out as follows. One μL of pPgps or pPctrl vector DNA was gently mixed into 40 μL of ΔREG(A5:YI) electrocompetent cells, which then were transferred to an electroporation cuvette with a 0.2 cM electrode gap. Electroporations were conducted using a Biorad Gene Pulser II (Biorad, Hercules, Calif.) with settings at 2.5 kV of potential, 400 ohms of resistance, and 25 μF of capacitance. Cells were recovered in 400 μL SOC media at 30° C. for 6-16 hours. The cells were then plated, 200 μL per plate, on LB medium containing 50 μg/ml kanamycin and incubated at 30° C. for 5-6 days.

After incubation, greenish colonies were observed on plates of ΔREG(A5:YI) transformed with pPctrl plasmid DNA. The colonies that appeared on plates of ΔREG(A5:YI) transformed with pPgps plasmid DNA appeared yellow. The yellow pigmentation was indicative of β-carotene production in ΔREG(A5:YI) expressing the A. fulgidus gps gene from pPgps.

Single yellow colonies were grown up in Sistrom's liquid media supplemented with vitamins, trace elements and 0.4% glucose as well as 50 μg/ml kanamycin, at 30° C. with shaking for 24-48 hours. Carotenoids were extracted and subjected to LCMS analysis as described above. Under the chromatography conditions used, β-carotene eluted at approximately 13.87-14.2 min. β-carotene standard (Sigma chemical, St. Louis, Mo.) was used to identify the peaks. The UV-Vis absorption spectra and the retention time using HPLC were used as diagnostic features for β-carotene identification in ΔREG(A5:YI) transformed with pPgps DNA, as well as the molecular ion and fragmentation patterns generated during mass spectrometry. Thus, the production of β-carotene was confirmed in ΔREG(A5:YI) expressing the A. fulgidus gps gene from pPgps.

### Example 6

Transformation of the β-carotene C4 ketolase (crtW) gene from Brevumdimonas aurantiacum and β-carotene hydroxylase (crtZ) from P. stewarii into the ΔREG(A5:Y1) strain of Rhodobacter with the gps gene from Archeoglubus fulgidus inserted into the chromosome: The following protocol describes the generation of an astaxanthin producing strain of R. sphaeroides using ΔREG(A5:YI), described above. See also Table 7 for further description of the strains and plasmids that were used in this example. Using the gene insertion method described by Higuchi, R., et al, Nucleic Acid Res. 16: 7351-7367, the crtA gene of ΔREG(A5:YI) was replaced by the gps gene from A. fulgidus to generate the strain ΔREG(A5:YI)(AA:gps). Electrocompetent cells ΔREG(A5:YI)(AA:gps) were generated as described above.

The construct pPgpsWZ was produced by cloning the crtw gene from B. aurantiacum, the crtZ gene from P.stewartii, and the gps gene from A fulgidus into the pPctrl plasmid using appropriate restriction enzymes. The construct pPWZ was produced by cloning the crtW gene from B. aurantiacum and the crtZ gene from P.stewartii into the pPctrl plasmid using appropriate restriction enzymes.

The pPWZ or pPgpsWZ constructs were electroporated into electrocompetent ΔREG(A5:YI)(AA:gps) as described earlier to generate ΔREG(A5:YI)(AA:gps)::pPWZ or ΔREG(A5:YI)(AA:gps)::pPgpsWZ, respectively. Transformation mixtures were plated out onto LB plates containing 50 μg/ml kanamycin. PCR analyses using PCR primers specific for crtZ were used to confirm the presence of the pPWZ or pPgpsWZ plasmids in ΔREG(A5:YI)(AA:gps).

Single colonies of ΔREG(A5:YI)(AA:gps)::pPWZ or ΔREG(A5:YI)(AA:gps)::pPgpsWZ were grown up in media supplemented with 50 μg/ml kanamycin as described earlier. Cell pellets were washed with distilled water and then carotenoids were extracted using acetone:methanol (7:2) at 30° C. for 30 mins with shaking at 225 rpm. Carotenoid analysis was performed using LCMS analysis described above. The UV-Vis absorption spectra and the retention time using HPLC were used as diagnostic features for astaxanthin identification in ΔREG(A5:YI)(AA:gps)::pPWZ and ΔREG(A5:YI)(AA:gps)::pPgpsWZ, as well as the molecular ion and fragmentation patterns generated during mass spectrometry. The production of astaxanthin was confirmed in both ΔREG(A5:YI)(AA:gps)::pPWZ and ΔREG(A5:YI)(AA:gps)::pPgpsWZ. Increased astaxanthin production was observed in ΔREG(A5:YI)(AA:gps)::pPgpsWZ.

### Example 7

Cloning and sequencing of a novel multifunctional Geranylgeranyl pyrophosphate synthase gene (ups) from Sulfolobus shibatae: Degenerate primer sequences MFGGPP1 (5′CCAYGAYGAYATWATGGA3′, SEQ ID NO:40) and MFGGPP2 (5′YTTYTTVCCYTYCCTAAT3′, SEQ ID NO:41) were designed based on conserved sequences in gps gene sequences from Sulfolobus solfotaricus and Sulfolobus acidocaldarius and synthesized by Integrated DNA Technologies (Coralville, Iowa). PCR was performed in a mastercycler gradient machine (Eppendorf) with genomic DNA from S. shibatae (ATCC Accession No. 51178, lot #1162977). Reaction conditions included five minutes at 96° C., followed by 30 cycles of denaturation at 94° C. for 30 sec., annealing at 50±10° C. for 60 sec., and extension at 72° C. for 90 sec., and a final 72° C. incubation for 10 min. An approximately 500-bp PCR product was obtained and cloned into the vector pC-BuntII-TOPO (Invitrogen Corp. Carlsbad, Calif.).

Independent clones were sequenced using the universal M13 forward and reverse primers. DNA sequencing was carried out at the AGAC, University of Minnesota, St. Paul, Minn. DNA sequence analysis of this PCR product indicated similarity to the gps genes from S. sulfotaricus and S. acidocaldarius. The Universal Genome Walker kit (Clontech) was used to obtain more of the gps gene sequence flanking the original PCR product from S. shibatae. Primers were synthesized based on the partial sequence and used for genome walking experiments.

The following strategy was used to completely sequence the S. shibatae gps gene. The ERWCRTS homolog was observed upstream of the S. sulfotaricus gps gene. The UDP-A-acetylglucosamine—Dolichyl-phosphate-N-acetylglucosamine phosphotransferase gene was present downstream of the gps gene in both S. sulfotaricus and S. acidocaldarius. Primers were designed based on the sequence of the two genes SsDolidn (5′ACAGCGTTGGACACTCAG 3′, SEQ ID NO:42) and SsERCRTup (5′GCGTCGATAATGGAAGTGAG 3′, SEQ ID NO:43) of the gps gene. An approximately 2 kb PCR product was amplified using the SsDolidn and SsERCRTup primers and genomic DNA from S. shibatae. This PCR product was cloned into the vector pC-BuntII-TOPO as described above and sequenced using the universal M13 forward and reverse primers. The nucleotide sequence of the gps gene from S. shibatae is presented in SEQ ID NO:44, and the amino acid sequence of the protein encoded by the gps gene is presented in SEQ ID NO:45.

### Example 8

### Down Regulation of crtC Gene Increases Carotenoid Production

Accession number for the carotenoid operon containing the crtC gene is AF195122. A 300-bp fragment from base position 274-574 internal to the crtC gene was deleted in order to create a non-functional crtC protein.

All restriction enzymes and T4 DNA ligase were obtained from New England Biolabs (Beverly, Mass.) unless otherwise indicated. All plasmid DNA preparations were done using QIAprep Spin Miniprep Kits or Qiagen Maxi Prep Kits and all gel purifications were done using QIAquick Gel Extraction Kits (Qiagen, Valencia, Calif.). Creation of a markerless crtC knockout using sacB selection in wild-type Rhodobacter sphaeroides 35053: A truncated crtC gene was cloned into pL01, a suicide vector in R. sphaeroides, to produce pL01 crtC. The pL01 vector contains a kanamycin resistance gene, a Bacillus subtilis sacB gene, an oriT sequence, a ColEI replicon, and a multiple cloning site (Lenz et al., 1994 J. Bacteriol. 176:4385-4393). The pL01 crtCplasmid was introduced into R. sphaeroides strain ATCC 35053, by conjugation with an E. coli (S17-1) donor. Kanamycin resistance was used to select for single-crossover events between the truncated crtC gene and the genomic crtC gene that resulted in incorporation of the pL01 crtC DNA into the genome. The presence of the sacB gene on the vector allowed for subsequent selection for the loss of vector DNA from the genome as expression of this gene in the presence of sucrose is lethal to E. coli and to R. sphaeroides under growth conditions of 5% and 15% sucrose, respectively. A portion of the double-crossover event that led to loss of the sacB gene contained the truncated crtC allele. This method of gene knockout is useful because no residual antibiotic resistance gene is left in the genome. A three-step PCR process was used to create a 300 bp in-frame deletion in the crtC gene. The crtC gene from R. sphaeroides strain 35053 was amplified by PCR using primers designed to introduce a Sac I restriction site at the beginning of the amplified fragment and a Xba I restriction site at the end of the amplified fragment.

The PCR reaction mix contained 0.2 mM each primer, 1× Expand reaction buffer, 2.5 ml DMSO, 0.2 mM each dNTP, 1×Expand/Pfu polymerase mix, and 1 ng of genomic DNA per 50 mL of reaction mix. PCR was conducted in a Perkin Elmer Geneamp 2400 programmed for an initial denaturation at 94° C. for 3 minutes followed by 25 cycles of denaturation for 30 sec at 94° C., annealing for 45 sec at 60° C., and extension for 2.5 min at 72° C., with a final extension at 72° C. for 7 minutes. A 1.83 KB reaction product was obtained by electrophoresis of a portion of the mixture (200 μl) through an 0.8% agarose gel in 1×TAE buffer and gel-purification.

The 20 nucleotides on the 3′ ends of each primer of this pair are located near the center of the crtC gene, 300 bases apart from each other, and facing towards the start and end of the gene. The 20 nucleotides on the 5′ end of each primer of this pair are the reverse complement of the 3′ end of the other primer in the pair. PCR of the two separate reactions was conducted as above, except that 0.05 ng of first round product per 50 μL of reaction mix was used as template. Also, an initial denaturation for 3 min at 94° C. was conducted followed by eight cycles of denaturation for 30 sec at 94° C., annealing for 30 sec at 60° C., and extension for 1 min 15 sec at 72° C.,; 22 cycles of denaturation for 30 sec at 94° C., annealing for 30 sec at 63° C., and extension for 1 min 15 sec at 72° C; and a final extension for 7 min at 72° C. Both PCR products (approximately 750 bp in length), were separated on a 0.8% agarose gel in 1×TAE buffer, excised, and gel purified.

The third round of PCR utilized the same primers and reaction mixture as the first round of PCR, except that a mixture of 10 ng of each second round fragment was used as template rather than genomic DNA (200 μL reaction). The PCR program used was also the same as that used in the first round of PCR, with the annealing time lengthened to 2 minutes. The 1.5 kB third-round product was separated on a 0.8% agarose gel in 1×TAE buffer and purified. The 1.3 kB PCR product (3 μg) was digested with Sacd and XbaI and purified using a QIAquick PCR Purification Kit.

Vector pL01 was prepared by digesting 3 μg of the vector with Sac I and Xba I, which were inactivated by heating to 65° C. for 20 minutes, and dephosphorylating using shrimp alkaline phosphatase (Roche). The dephosphorylated vector was gel purified on a 1% TAE-agarose gel.

SacI and XbaI digested vector DNA (66 ng) was ligated with 80 ng of the digested third-round PCR product at room temperature for 5 min using Rapid DNA ligase system (Roche). A portion of the ligation mixture (1 μl) was electroporated into 40 μL of E. coli ElectroMAXTM DH510BTM electrocompetent cells (Life Technologies). Electroporated cells were plated on LB media containing 50 mg/mL kanamycin (LBK50). Individual colonies were picked and patched to fresh LBK50 plates and simultaneously resuspended in 25 μl distilled water (D/W) and heated at 95° C. for 10 min to lyse the cells and release the DNA. Colonies with insert were identified using a PCR screen that was identical to the first round of PCR described earlier.

Donor E. coli colonies for conjugation were prepared by electroporating 1 μl of plasmid DNA into electrocompetent S17-1 cells. Electroporated cells were plated on LB media containing 25 μg/mL of kanamycin, 25 μg/mL of streptomycin, and 25 μg/mL of spectinomycin (LBKSMST). Single colonies were used to start cultures for plasmid DNA isolation and use in conjugation. These colonies also were plated on LB media containing 5% sucrose and 25 μg/mL of kanamycin to ensure that the sacB gene was still functional. Only colonies that showed lethality on the sucrose media were used in conjugation. The presence of the correct insert size was confirmed using a PCR screen that was identical to the first round of PCR described above.

Growing cultures of R. sphaeroides strain 35053 were sub-cultured, using 1/5 and 1/10 volumes of inoculum, in 5 mL Sistrom's media supplemented with 20% LB and grown at 30° C. for 12 hours. The S17-1 donor colonies were grown in LBKSMST media at 37° C. for 12 hours. An aliquot of each culture (1.5-3.0 mL) was pelleted and the pellets were washed four times with LB media. Relative pellet size was estimated and approximately 2 volumes of 35053 cells were used to 1 volume of S17-1 cells. The cell mixture was pelleted, resuspended in 20 μL of LB media, spotted on an LB plate, and incubated at 30° C. for 7 -15 hours. The cells then were scraped off the surface of the plate, resuspended in 1.5 mL of Sistrom's salts, and 200 μL of resuspended cells were plated on each of seven plates of Sistrom's with 25 μg/mL kanamycin (SISK25) media. Colonies that grew on the plates after approximately 10 days, representing proposed single-crossover events, were streaked to new plates of the same media. Upon growth, single colonies were streaked on LB with 25 μg/mL kanamycin (LBK25) media. Purified colonies were patched to Sistrom's media supplemented with 1×LB, 15% sucrose, 0.5% DMSO (v/v), and 25 μg/mL kanamycin (SisLBKI5%SucDMSO). These were grown in an anaerobic chamber (Becton Dickinson, Sparks, Md.) at 30° C. for 5 days to check for lethality of the sacB gene in the proposed single-crossover events.

Concurrently, the cultures had been patched to SisLB media containing 15% sucrose and 0.5% DMSO (v/v) without kanamycin (SisLB 1 5% SucDMSO). Colonies were purified from these cultures and were tested by PCR to show that they contained the truncated crtC allele. Potential double crossovers also were streaked on LBK25 plates to confirm sensitivity to kanamycin.

To assay for the effect of the deletion on carotenoid production in R. sphaeroides the Rhodobacter sphaeroides were grown using the following shake flask protocol: Cultures of R. sphaeroides ATCC 35053 with various inserted genes or knockouts were grown in 5 mL culture tubes containing Sistrom's media with 4 g/L glucose (Sistrom, 1962. J. Gen. Microb. 28:607-616). The cultures were incubated for 48 to 72 hours at 30° C. with 250 rpm shaking in a New Brunswick Innova Shaker. A 1.6 mL aliquot of each 5 mL culture was removed from the culture tube and added to 150 ml of Tris urea medium with 0.8% yeast extract (Sigma Chemical Co., St. Louis, Mo.) in a 500 ml baffled shake flask. Tris urea medium is a modification of Sistrom's medium in which the ammonium sulfate has been removed and 50 mM Tris HCI, 1.6 g/L urea, and 10 g/L glucose have been added. The flask was then incubated for 72 to 84 hours at 30° C. with shaking at 90 rpms in a New Brunswick Innova Shaker. The entire contents of the flask were removed at the end of the incubation period for carotenoid analysis.

Analysis of carotenoid from R. sphaeroides wild type and crtC deletion strain (crtC deletion): Samples with a volume of 20 mL were harvested by centrifugation at 3500 rpm for 10 min. The sample was washed once in 20 mL of distilled water and resuspended in an equal volume of distilled water. A ten mL sample was centrifuged in a separate tube at 3500 rpm for 10 min., resuspended in approximately one ml of distilled water, and poured into a tared pan for dry cell weight analysis. The sample was dried for 24 hours at 100° C. and the dry cell weight (DCW)/mL of culture was calculated. For extraction, a volume of 0.75 mL to 1.5 mL of culture was added to a 1.8 mL-microfuge tube and centrifuged at 10,000 rpm for 3 min in an IEC MicroMax microfuge. The supernatant was discarded and the pellet was completely resuspended in 1.0 mL of acetone: methanol (7:2) and stored at room temperature in the dark for 30 min. The sample was mixed once during this incubation. After incubation, the sample was centrifuged at 10,000 rpm for 3 min. and the extract (supernatant) was collected. Samples were stored at −20° C. if analysis was not performed immediately. Carotenoid extracts were analyzed on a spectrophotometer, scanning in the range of 350 nm to 800 nm For Spheroidenone analysis: The amount of carotenoid in mg/100 mls of culture was calculated using the following equation: spheroidenone (mg)/100 mls culture=((OD480−(0.0816*OD 770))*0.484)/Vol. of original sample extracted. From mg of spheroidenone/100 mls of culture, the amount of spheroidenone/mg of DCW (dry well weight) was calculated using the DCW number as the conversion factor. Care was taken to correct for any dilutions made to the sample being analyzed. Concentration for spheroidenone was calculated using an extinction coefficient (E 1% 1 cm) of 2120 (E.A. Shneour Biochemica et Biophysica Acta, 62 (1962) 534-540. Carotenoid Pigment Conversion in Rhodopseudomonas spheroides)

For Neurosporene analysis: The amount of carotenoid in mg/100 mls of culture was calculated using the following equation: neurosporene (mg)/100 mls culture=((OD440−(0.1138*OD 770))*0.343)/Vol. of original sample extracted. From mg of neurosporene/100 mls of culture, the amount of neurosporene/mg of DCW was calculated using the DCW number as the conversion factor. Care was taken to correct for any dilutions made to the sample being analyzed. Concentration for neurosporene was calculated using an extinction coefficient (E 1% 1 cm) of 2918 (Carotenoids Volume 1B Spectroscopy, Edited by G. Britton, S. Liaaen-Jensen and H. Pfander pg 60.).

The results are provided below in Table 8.

TABLE 8

| Strain | CoQ₁₀ | Bchl-ppm | Crt-ppm | Total ppm |
|---|---|---|---|---|
| wild Type | 5499 | 25380 | 5618 | 36497 |
| CrtC | 5583 | 23033 | 5180 | 33796 |
| deletion | | | | |

CoQ₁₀ = Coenzyme Q 10,
Bchl = bacteriochlorophyll, and
Crt = carotenoid

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of increasing carotenoid production in R. sphaeroides, consisting of:
down-regulating the expression of the carotenoid biosynthetic pathway gene crtC.

2. The method according to claim 1, further comprising down regulating one or more regulatory genes selected from the group consisting of ppsR, ccoN and/or aerR.

3. The method according to claim 1, wherein one or more carotenoids selected from the group consisting of lycopene, beta-carotein, zeaxanthin, decapreoxanthin, lutein, and/or astaxanthin are produced.


SEQUENCE LISTING (47 sequences)

SEQ ID NO: 1 - DNA, 1296 bp, Pantoea stewartii
atgagccattttgcggtgatcgcaccgccctttttcagccatgttcgcgctctgcaaaaccttgctcaggaattagtggcccgcggtcatcgtgttacgttttttcagcaacatgactgcaaagcgctggtaacgggcagcgatatcggattccagaccgtcggactgcaaacgcatcctcccggttccttatcgcacctgctgcacctggccgcgcacccactcggaccctcgatgttacgactgatcaatgaaatggcacgtaccagcgatatgctttgccgggaactgcccgccgcttttcatgcgttgcagatagagggcgtgatcgttgatcaaatggagccggcaggtgcagtagtcgcagaagcgtcaggtctgccgtttgtttcggtggcctgcgcgctgccgctcaaccgcgaaccgggtttgcctctggcggtgatgcctttcgagtacggcaccagcgatgcggctcgggaacgctataccaccagcgaaaaaatttatgactggctgatgcgacgtcacgatcgtgtgatcgcgcatcatgcatgcagaatgggtttagccccgcgtgaaaaactgcatcattgtttttctccactggcacaaatcagccagttgatccccgaactggattttccccgcaaagcgctgccagactgctttcatgcggttggaccgttacggcaaccccaggggacgccggggtcatcaacttcttattttccgtccccggacaaaccccgtatttttgcctcgctgggcaccctgcagggacatcgttatggcctgttcaggaccatcgccaaagcctgcgaagaggtggatgcgcagttactgttggcacactgtggcggcctctcagccacgcaggcaggtgaactggcccggggcggggacattcaggttgtggattttgccgatcaatccgcagcactttcacaggcacagttgacaatcacacatggtgggatgaatacggtactggacgctattgcttcccgcacaccgctactggcgctgccgctggcatttgatcaacctggcgtggcatcacgaattgtttatcatggcatcggcaagcgtgcgtctcggtttactaccagccatgcgctggcgcggcagattcgatcgctgctgactaacaccgattacccgcagcgtatgacaaaaattcaggccgcattgcgtctggcaggcggcacaccagccgccgccgatattgttgaacaggcgatgcggacctgtcagccagtactcagtgggcaggattatgcaaccgcactatga

SEQ ID NO: 2 - protein, 431 aa, Pantoea stewartii
MSHFAVIAPPFFSHVRALQNLAQELVARGHRVTFFQQHDCKALVTGSDIGFQTVGLQTHPPGSLSHLLHLAAHPLGPSMLRLINEMARTSDMLCRELPAAFHALQIEGVIVDQMEPAGAVVAEASGLPFVSVACALPLNREPGLPLAVMPFEYGTSDAARERYTTSEKIYDWLMRRHDRVIAHHACRMGLAPREKLHHCFSPLAQISQLIPELDFPRKALPDCFHAVGPLRQPQGTPGSSTSYFPSPDKPRIFASLGTLQGHRYGLFRTIAKACEEVDAQLLLAHCGGLSATQAGELARGGDIQVVDFADQSAALSQAQLTITHGGMNTVLDAIASRTPLLALPLAFDQPGVASRIVYHGIGKRASRFTTSHALARQIRSLLTNTDYPQRMTKIQAALRLAGGTPAAADIVEQAMRTCQPVLSGQDYATAL

SEQ ID NO: 3 - DNA, 1149 bp, Pantoea stewartii
atgcaaccgcactatgatctcattctggtcggtgccggtctggctaatggccttatcgcgctccggcttcagcaacagcatccggatatgcggatcttgcttattgaggcgggtcctgaggcgggagggaaccatacctggtcctttcacgaagaggatttaacgctgaatcagcatcgctggatagcgccgcttgtggtccatcactggcccgactaccaggttcgtttcccccaacgccgtcgccatgtgaacagtggctactactgcgtgacctcccggcatttcgccgggatactccggcaacagtttggacaacatttatggctgcataccgcggtttcagccgttcatgctgaatcggtccagttagcggatggccggattattcatgccagtacagtgatcgacggacggggttacacgcctgattctgcactacgcgtaggattccaggcatttatcggtcaggagtggcaactgagcgcgccgcatggtttatcgtcaccgattatcatggatgcgacggtcgatcagcaaaatggctaccgctttgtttataccctgccgctttccgcaaccgcactgctgatcgaagacacacactacattgacaaggctaatcttcaggccgaacgggcgcgtcagaacattcgcgattatgctgcgcgacagggttggccgttacagacgttgctgcgggaagaacagggtgcattgcccattacgttaacgggcgataatcgtcagttttggcaacagcaaccgcaagcctgtagcggattacgcgccgggctgtttcatccgacaaccggctactccctaccgctcgcggtggcgctggccgatcgtctcagcgcgctggatgtgtttacctcttcctctgttcaccagacgattgctcactttgcccagcaacgttggcagcaacaggggtttttccgcatgctgaatcgcatgttgtttttagccggaccggccgagtcacgctggcgtgtgatgcagcgtttctatggcttacccgaggatttgattgcccgcttttatgcgggaaaactcaccgtgaccgatcggctacgcattctgagcggcaagccgcccgttcccgttttcgcggcattgcaggcaattatgacgactcatcgttga

SEQ ID NO: 4 - protein, 382 aa, Pantoea stewartii
MQPHYDLILVGAGLANGLIALRLQQQHPDMRILLIEAGPEAGGNHTWSFHEEDLTLNQHRWIAPLVVHHWPDYQVRFPQRRRHVNSGYYCVTSRHFAGILRQQFGQHLWLHTAVSAVHAESVQLADGRIIHASTVIDGRGYTPDSALRVGFQAFIGQEWQLSAPHGLSSPIIMDATVDQQNGYRFVYTLPLSATALLIEDTHYIDKANLQAERARQNIRDYAARQGWPLQTLLREEQGALPITLTGDNRQFWQQQPQACSGLRAGLFHPTTGYSLPLAVALADRLSALDVFTSSSVHQTIAHFAQQRWQQQGFFRMLNRMLFLAGPAESRWRVMQRFYGLPEDLIARFYAGKLTVTDRLRILSGKPPVPVFAALQAIMTTHR

SEQ ID NO: 5 - DNA, 912 bp, Pantoea stewartii
atgatggtctgcgcaaaaaaacacgttcaccttactggcatttcggctgagcagttgctggctgatatcgatagccgccttgatcagttactgccggttcagggtgagcgggattgtgtgggtgccgcgatgcgtgaaggcacgctggcaccgggcaaacgtattcgtccgatgctgctgttattaacagcgcgcgatcttggctgtgcgatcagtcacgggggattactggatttagcctgcgcggttgaaatggtgcatgctgcctcgctgattctggatgatatgccctgcatggacgatgcgcagatgcgtcgggggcgtcccaccattcacacgcagtacggtgaacatgtggcgattctggcggcggtcgctttactcagcaaagcgtttggggtgattgccgaggctgaaggtctgacgccgatagccaaaactcgcgcggtgtcggagctgtccactgcgattggcatgcagggtctggttcagggccagtttaaggacctctcggaaggcgataaaccccgcagcgccgatgccatactgctaaccaatcagtttaaaaccagcacgctgttttgcgcgtcaacgcaaatggcgtccattgcggccaacgcgtcctgcgaagcgcgtgagaacctgcatcgtttctcgctcgatctcggccaggcctttcagttgcttgacgatcttaccgatggcatgaccgataccggcaaagacatcaatcaggatgcaggtaaatcaacgctggtcaatttattaggctcaggcgcggtcgaagaacgcctgcgacagcatttgcgcctggccagtgaacacctttccgcggcatgccaaaacggccattccaccacccaactttttattcaggcctggtttgacaaaaaactcgctgccgtcagttaa

SEQ ID NO: 6 - protein, 303 aa, Pantoea stewartii
MMVCAKKHVHLTGISAEQLLADIDSRLDQLLPVQGERDCVGAAMREGTLAPGKRIRPMLLLLTARDLGCAISHGGLLDLACAVEMVHAASLILDDMPCMDDAQMRRGRPTIHTQYGEHVAILAAVALLSKAFGVIAEAEGLTPIAKTRAVSELSTAIGMQGLVQGQFKDLSEGDKPRSADAILLTNQFKTSTLFCASTQMASIAANASCEARENLHRFSLDLGQAFQLLDDLTDGMTDTGKDINQDAGKSTLVNLLGSGAVEERLRQHLRLASEHLSAACQNGHSTTQLFIQAWFDKKLAAVS

SEQ ID NO: 7 - DNA, 1479 bp, Pantoea stewartii
atgaaaccaactacggtaattggtgcgggctttggtggcctggcactggcaattcgtttacaggccgcaggtattcctgttttgctgcttgagcagcgcgacaagccgggtggccgggcttatgtttatcaggagcagggctttacttttgatgcaggccctaccgttatcaccgatcccagcgcgattgaagaactgtttgctctggccggtaaacagcttaaggattacgtcgagctgttgccggtcacgccgttttatcgcctgtgctgggagtccggcaaggtcttcaattacgataacgaccaggcccagttagaagcgcagatacagcagtttaatccgcgcgatgttgcgggttatcgagcgttccttgactattcgcgtgccgtattcaatgagggctatctgaagctcggcactgtgccttttttatcgttcaaagacatgcttcgggccgcgccccagttggcaaagctgcaggcatggcgcagcgtttacagtaaagttgccggctacattgaggatgagcatcttcggcaggcgttttcttttcactcgctcttagtgggggggaatccgtttgcaacctcgtccatttatacgctgattcacgcgttagaacgggaatggggcgtctggtttccacgcggtggaaccggtgcgctggtcaatggcatgatcaagctgtttcaggatctgggcggcgaagtcgtgcttaacgcccgggtcagtcatatggaaaccgttggggacaagattcaggccgtgcagttggaagacggcagacggtttgaaacctgcgcggtggcgtcgaacgctgatgttgtacatacctatcgcgatctgctgtctcagcatcccgcagccgctaagcaggcgaaaaaactgcaatccaagcgtatgagtaactcactgtttgtactctattttggtctcaaccatcatcacgatcaactcgcccatcataccgtctgttttgggccacgctaccgtgaactgattcacgaaatttttaaccatgatggtctggctgaggatttttcgctttatttacacgcaccttgtgtcacggatccgtcactggcaccggaagggtgcggcagctattatgtgctggcgcctgttccacacttaggcacggcgaacctcgactgggcggtagaaggaccccgactgcgcgatcgtatttttgactaccttgagcaacattacatgcctggcttgcgaagccagttggtgacgcaccgtatgtttacgccgttcgatttccgcgacgagctcaatgcctggcaaggttcggccttctcggttgaacctattctgacccagagcgcctggttccgaccacataaccgcgataagcacattgataatctttatctggttggcgcaggcacccatcctggcgcgggcattcccggcgtaatcggctcggcgaaggcgacggcaggcttaatgctggaggacctgatttga

SEQ ID NO: 8 - protein, 492 aa, Pantoea stewartii
MKPTTVIGAGFGGLALAIRLQAAGIPVLLLEQRDKPGGRAYVYQEQGFTFDAGPTVITDPSAIEELFALAGKQLKDYVELLPVTPFYRLCWESGKVFNYDNDQAQLEAQIQQFNPRDVAGYRAFLDYSRAVFNEGYLKLGTVPFLSFKDMLRAAPQLAKLQAWRSVYSKVAGYIEDEHLRQAFSFHSLLVGGNPFATSSIYTLIHALEREWGVWFPRGGTGALVNGMIKLFQDLGGEVVLNARVSHMETVGDKIQAVQLEDGRRFETCAVASNADVVHTYRDLLSQHPAAAKQAKKLQSKRMSNSLFVLYFGLNHHHDQLAHHTVCFGPRYRELIHEIFNHDGLAEDFSLYLHAPCVTDPSLAPEGCGSYYVLAPVPHLGTANLDWAVEGPRLRDRIFDYLEQHYMPGLRSQLVTHRMFTPFDFRDELNAWQGSAFSVEPILTQSAWFRPHNRDKHIDNLYLVGAGTHPGAGIPGVIGSAKATAGLMLEDLI

SEQ ID NO: 9 - DNA, 893 bp, Pantoea stewartii
ccatggcggttggctcgaaaagctttgcgactgcatcgacgcttttcgacgccaaaacccgtcgcagcgtgctgatgctttacgcatggtgccgccactgcgacgacgtcattgacgatcaaacactgggctttcatgccgaccagccctcttcgcagatgcctgagcagcgcctgcagcagcttgaaatgaaaacgcgtcaggcctacgccggttcgcaaatgcacgagcccgcttttgccgcgtttcaggaggtcgcgatggcgcatgatatcgctcccgcctacgcgttcgaccatctggaaggttttgccatggatgtgcgcgaaacgcgctacctgacactggacgatacgctgcgttattgctatcacgtcgccggtgttgtgggcctgatgatggcgcaaattatgggcgttcgcgataacgccacgctcgatcgcgcctgcgatctcgggctggctttccagttgaccaacattgcgcgtgatattgtcgacgatgctcaggtgggccgctgttatctgcctgaaagctggctggaagaggaaggactgacgaaagcgaattatgctgcgccagaaaaccggcaggccttaagccgtatcgccgggcgactggtacgggaagcggaaccctattacgtatcatcaatggccggtctggcacaattacccttacgctcggcctgggccatcgcgacagcgaagcaggtgtaccgtaaaattggcgtgaaagttgaacaggccggtaagcaggcctgggatcatcgccagtccacgtccaccgccgaaaaattaacgcttttgctgacggcatccggtcaggcagttacttcccggatgaagacgtatccaccccgtcctgctcatctctggcagcgcccgatctag

SEQ ID NO: 10 - protein, 296 aa, Pantoea stewartii
MAVGSKSFATASTLFDAKTRRSVLMLYAWCRHCDDVIDDQTLGFHADQPSSQMPEQRLQQLEMKTRQAYAGSQMHEPAFAAFQEVAMAHDIAPAYAFDHLEGFAMDVRETRYLTLDDTLRYCYHVAGVVGLMMAQIMGVRDNATLDRACDLGLAFQLTNIARDIVDDAQVGRCYLPESWLEEEGLTKANYAAPENRQALSRIAGRLVREAEPYYVSSMAGLAQLPLRSAWAIATAKQVYRKIGVKVEQAGKQAWDHRQSTSTAEKLTLLLTASGQAVTSRMKTYPPRPAHLWQRPI

SEQ ID NO: 11 - DNA, 528 bp, Pantoea stewartii
atgttgtggatttggaatgccctgatcgtgtttgtcaccgtggtcggcatggaagtggttgctgcactggcacataaatacatcatgcacggctggggttggggctggcatctttcacatcatgaaccgcgtaaaggcgcatttgaagttaacgatctctatgccgtggtattcgccattgtgtcgattgccctgatttacttcggcagtacaggaatctggccgctccagtggattggtgcaggcatgaccgcttatggtttactgtattttatggtccacgacggactggtacaccagcgctggccgttccgctacataccgcgcaaaggctacctgaaacggttatacatggcccaccgtatgcatcatgctgtaaggggaaaagagggctgcgtgtcctttggttttctgtacgcgccaccgttatctaaacttcaggcgacgctgagagaaaggcatgcggctagatcgggcgctgccagagatgagcaggacggggtggatacgtcttcatccgggaagtaa

SEQ ID NO: 12 - protein, 175 aa, Pantoea stewartii
MLWIWNALIVFVTVVGMEVVAALAHKYIMHGWGWGWHLSHHEPRKGAFEVNDLYAVVFAIVSIALIYFGSTGIWPLQWIGAGMTAYGLLYFMVHDGLVHQRWPFRYIPRKGYLKRLYMAHRMHHAVRGKEGCVSFGFLYAPPLSKLQATLRERHAARSGAARDEQDGVDTSSSGK

SEQ ID NO: 13 - DNA, 29 bp, Artificial Sequence
  Primer
atyatgcacggctggggwtggsgmtggca

SEQ ID NO: 14 - DNA, 31 bp, Artificial Sequence
  Primer
ggccarcgytgatgcaccagmccgtcrtgca

SEQ ID NO: 15 - DNA, 26 bp, Artificial Sequence
  Primer
ctgatgctctaygcctggtgccgcca

SEQ ID NO: 16 - DNA, 23 bp, Artificial Sequence
  Primer
tcgcgrgcratrttsgtcarctg

SEQ ID NO: 17 - DNA, 20 bp, Artificial Sequence
  Primer
atbmtsatggaygcsacsgt

SEQ ID NO: 18 - DNA, 20 bp, Artificial Sequence
  Primer
ytratcgargayacgcrcta

SEQ ID NO: 19 - DNA, 20 bp, Artificial Sequence
  Primer
rsggcagygaatagccrgtg

SEQ ID NO: 20 - DNA, 25 bp, Artificial Sequence
  Primer
aacagcatscgrttcagcakgcgsa

SEQ ID NO: 21 - DNA, 20 bp, Artificial Sequence
  Primer
ccgacggtkatcaccgatcc

SEQ ID NO: 22 - DNA, 19 bp, Artificial Sequence
  Primer
ctgcgccsaccaggtagag

SEQ ID NO: 23 - DNA, 24 bp, Artificial Sequence
  Primer
ctygacgayatgccctgcatggac

SEQ ID NO: 24 - DNA, 24 bp, Artificial Sequence
  Primer
gtcgatttwccsgcgtcctkattg

SEQ ID NO: 25 - DNA, 30 bp, Artificial Sequence
  Primer
ggccgaattccaacgatgctctggcagtta

SEQ ID NO: 26 - DNA, 30 bp, Artificial Sequence
  Primer
ggccagatctacttcaggcgacgctgagag

SEQ ID NO: 27 - DNA, 30 bp, Artificial Sequence
  Primer
ggccagatcttacgcgcgggtaaagccaat

SEQ ID NO: 28 - DNA, 30 bp, Artificial Sequence
  Primer
ggcctctagaattaccgcgtggttctgaag

SEQ ID NO: 29 - DNA, 30 bp, Artificial Sequence
  Primer
ggcctctagatctgtacgcgccaccgttat

SEQ ID NO: 30 - DNA, 27 bp, Artificial Sequence
  Primer
catcggtaagatcgtcaagcaactgaa

SEQ ID NO: 31 - DNA, 27 bp, Artificial Sequence
  Primer
gatttacctgcatcctgattgatgtct

SEQ ID NO: 32 - DNA, 27 bp, Artificial Sequence
  Primer
atgtataaccgtttcaggtagcctttg

SEQ ID NO: 33 - DNA, 27 bp, Artificial Sequence
  Primer
aatacagtaaaccataagcggtcatgc

SEQ ID NO: 34 - DNA, 18 bp, Artificial Sequence
  Primer
ttcatcatcgcgcatgac

SEQ ID NO: 35 - DNA, 18 bp, Artificial Sequence
  Primer
agrtgrtgytcgtgrtga

SEQ ID NO: 36 - DNA, 21 bp, Artificial Sequence
  Primer
gcggcataggctagattgaag

SEQ ID NO: 37 - DNA, 20 bp, Artificial Sequence
  Primer
gcgagttccttctcacctat

SEQ ID NO: 38 - DNA, 735 bp, Brevundimonas aurantiaca
atgaccgccgccgtcgccgagccacgcaccgtcccgcgccagacctggatcggtctgaccctggcgggaatgatcgtggcgggatgggcggttctgcatgtctacggcgtctattttcaccgatgggggccgttgaccctggtgatcgccccggcgatcgtggcggtccagacctggttgtcggtcggccttttcatcgtcgcccatgacgccatgtacggctccctggcgccgggacggccgcggctgaacgccgcagtcggccggctgaccctggggctctatgcgggcttccgcttcgatcggctgaagacggcgcaccacgcccaccacgccgcgcccggcacggccgacgacccggattttcacgccccggcgccccgcgccttccttccctggttcctgaacttctttcgcacctatttcggctggcgcgagatggcggtcctgaccgccctggtcctgatcgccctcttcggcctgggggcgcggccggccaatctcctgaccttctgggccgcgccggccctgctttcagcgcttcagctcttcaccttcggcacctggctgccgcaccgccacaccgaccagccgttcgccgacgcgcaccacgcccgcagcagcggctacggccccgtgctttccctgctcacctgtttccacttcggccgccaccacgaacaccatctgagcccctggcggccctggtggcgtctgtggcgcggcgagtcttga

SEQ ID NO: 39 - protein, 244 aa, Brevundimonas aurantiaca
MTAAVAEPRTVPRQTWIGLTLAGMIVAGWAVLHVYGVYFHRWGPLTLVIAPAIVAVQTWLSVGLFIVAHDAMYGSLAPGRPRLNAAVGRLTLGLYAGFRFDRLKTAHHAHHAAPGTADDPDFHAPAPRAFLPWFLNFFRTYFGWREMAVLTALVLIALFGLGARPANLLTFWAAPALLSALQLFTFGTWLPHRHTDQPFADAHHARSSGYGPVLSLLTCFHFGRHHEHHLSPWRPWWRLWRGES

SEQ ID NO: 40 - DNA, 18 bp, Artificial Sequence
  Primer
ccaygaygayatwatgga

SEQ ID NO: 41 - DNA, 18 bp, Artificial Sequence
  Primer
yttyttvccytycctaat

SEQ ID NO: 42 - DNA, 18 bp, Artificial Sequence
  Primer
acagcgttggacactcag

SEQ ID NO: 43 - DNA, 20 bp, Artificial Sequence
  Primer
gcgtcgataatggaagtgag

SEQ ID NO: 44 - DNA, 1496 bp, Sulfolobus shibatae
ttaccagtgttaaaaagtgctatagaaggtaaggaaagtttagaacaattctttagaaagataatatttgaattgaaggccgccatgatgcttactggttctaaagacgttgatgcgttaaagaagaccagtattgttattttaggtaaacttaaagagtgggcagaatatagggggataaatttatctatatacgagaaagttagaaagagagaataaaatgagtgacgaattaagttcgtattttaatgatatagttaacaatgtaaattttcatataaaaaattttgtaaagagcaatgttagaacgcttgaggaagcatcgtttcatttatttacagctgggggcaaaagacttagacccttaattctggtttcatcgtcagacttaattggcggggacaggcaaagggcatataaggcagcagctgccgtggagattcttcacaactttactctagttcatgacgatataatggatagggattacctaagaagaggattaccaactgttcatgtaaagtggggtgaaccaatggcaatacttgcaggtgattacttacacgccaaggcttttgaagccttaaatgaggctctaaaaggtcttgacgggaatacgttttataaggctttttccgtatttattaattctattgagataatatcggaaggtcaagcaatggatatgtcatttgaaaatagagtagatgtaactgaggaagagtacatgcaaatgataaaaggaaagactgcgatgctattttcatgttctgctgcattaggcggtataattaacaaggctagcgatgatataattaaaaatttagtcgaatatggattaaatctaggcatatcattccaaatagtggatgatatcttaggaattattggagaccaaaaggaattagggaaaccagtttacagtgatattagggaaggtaagaaaacaattcttgttataaaaactttaagtgaagctactgacgatgaaaagaaaattctggtttctacgcttgggaatagggaggctaaaaaggacgatcttgagagagcgtcggaaataataaggaagtattcattgcaatatgcatacaatttagctaaaaagtactcagatcttgcattagaacatttgcgtaaaattccagtttacaatgaaactgctgaaaaggctttaaaatatctagcgcagtttaccattgaaaggagaaagtaaatgagcatatcagggatattgctttcaatttttatatcctttttcataagctatattacaacagtctgggtaataagacaggcaaaaaagagtgggcttgtaggtaaggatgtaaataaaccagataaaccggaaataccactaatgggtgggataagtataatagccgggtttatagcgggatccttctccttattactaactgatgtaagaagtgagcgagtaattccatctgtaatactctcctcattgcttatagcatttcttggactattagatg

SEQ ID NO: 45 - protein, 331 aa, Sulfolobus shibatae
MSDELSSYFNDIVNNVNFHIKNFVKSNVRTLEEASFHLFTAGGKRLRPLILVSSSDLIGGDRQRAYKAAAAVEILHNFTLVHDDIMDRDYLRRGLPTVHVKWGEPMAILAGDYLHAKAFEALNEALKGLDGNTFYKAFSVFINSIEIISEGQAMDMSFENRVDVTEEEYMQMIKGKTAMLFSCSAALGGIINKASDDIIKNLVEYGLNLGISFQIVDDILGIIGDQKELGKPVYSDIREGKKTILVIKTLSEATDDEKKILVSTLGNREAKKDDLERASEIIRKYSLQYAYNLAKKYSDLALEHLRKIPVYNETAEKALKYLAQFTIERRK

SEQ ID NO: 46 - DNA, 20 bp, Artificial Sequence
  Exemplary motif
aggtcgtgtactgtcagtca

SEQ ID NO: 47 - DNA, 20 bp, Artificial Sequence
  Exemplary motif
acgtggtgaactgccagtga